(19) United States
(12) Patent Application Publication
(10) Pub. No.: US 2022/0296814 A1
(43) Pub. Date: Sep. 22, 2022

(54) Medicament Container with Electronic Identifier

(71) Applicant: Sanofi, Paris (FR)

(72) Inventor: Michael Helmer, Frankfurt am Main (DE)

(21) Appl. No.: 17/634,680

(22) PCT Filed: Aug. 25, 2020
(86) PCT No.: PCT/EP2020/073673

§ 371 Date: Feb. 11, 2022

(30) Foreign Application Priority Data
Aug. 26, 2019 (EP) 19306029.0
May 29, 2020 (EP) 20315277.2

(51) Int. Cl. A61M 5/31

(52) U.S. Cl. CPC A61M 5/3129; A61M 2205/3592; A61M 2205/60

(57) ABSTRACT

The present disclosure relates to a medicament container (100; 200) for a liquid medicament, the medicament container comprising:—a barrel (101; 201) defining a longitudinal direction (2, 3) and comprising an interior volume (105) to accommodate the liquid medicament, the barrel (101; 201) comprising a distal end (106) and a proximal end (108), the barrel (101; 201) comprising an outlet (102; 202) for the liquid medicament at the distal end (106; 206),—a mount (110; 210) for an electronic identifier (150),—an electronic identifier (150) engaged with the mount (110; 210) and attached to the barrel (101; 201) via the mount (110; 210).

### FIELD

The present disclosure relates to the field medicament containers and in particular to the field of so-called primary medicament containers, i.e. medicament containers that are in direct contact with a medicament or drug. In some aspects the disclosure relates to medicament containers associated with an identifier. Moreover, the disclosure relates to an injection device equipped with such a medicament container.

### BACKGROUND

Drug delivery devices, such as injection devices or infusion devices are configured for setting and dispensing a single dose or multiple doses of a liquid medicament.

Injection or infusion devices, e.g. in form of pen-type injectors or infusion pumps, have to meet a number of user-specific requirements. For instance, with patient's suffering chronic diseases, such as diabetes, the patient may be physically infirm and may also have impaired vision.

Suitable injection or infusion devices especially intended for home medication therefore need to be robust in construction and should be easy to use. Furthermore, manipulation and general handling of the device and its components should be intelligible and easy understandable. Moreover, the dose setting as well as dose dispensing procedure must be easy to operate and has to be unambiguous.

Typically, injection or infusion devices comprise a housing with a mechanical interface configured to releasably or non-releasably engage with a medicament container. Some injection or infusion devices comprise a container holder configured to accommodate a medicament container. With such injection or infusion devices the medicament container is configured to be inserted into the housing of the injection device or infusion device.

In the area of medical device design, manufacture, assembly, distribution and sale of medical medicament containers it may be important to have the ability to track the history of individual medicament containers or devices equipped with such containers. Hence, it is desirable to provide a tractability or traceability of individual medicament or medicament containers as well as of lots of medicament containers from an early stage of manufacturing through the end use of the medicament container or of a respective drug delivery device using such a medicament container.

Moreover, acquisition and retrieving of data being indicative of at least one of numerous available ambient parameters of a medicament container and/or of a drug delivery device and data being indicative of the administering of subsequent doses of the medicament has gained attraction and has turned into an emerging field of investigation.

Medicament containers of primary packaging type, i.e. medicament containers that have a direct mechanical contact with the medicament have to be pharmaceutically inert regard to the medicament over the entire lifetime of the medicament. In view of this, only a few materials or material combinations exist that are compliant with existing regulatory provisions for use in a primary packaging.

A direct labeling or marking of individual medicament containers configured for an automatic processing, data acquisition, data processing as well as data retrieval is quite difficult. The limited number of materials generally available for the manufacturing of a primary package and/or of a medicament container do not provide an easy integration of electronically readable electronic identifiers. A direct embedding of an electronic identifier in the bulk of material forming or constituting the primary container or medicament container is difficult to achieve because electronically implemented identifiers are quite sensitive to heat.

In this regard the present disclosure aims to provide an improved medicament container equipped with an electronic identifier for purposes of tracking the medicament container from an early stage of manufacturing until the end of lifetime. The medicament container should be easy to implement at low or moderate costs. The medicament container should provide integration of a number of components and functions of a drug delivery device. The medicament container should enable the replacement and substitution of numerous components of a drug delivery device. In particular, the medicament container should allow to reduce the number of individual parts or components the drug delivery device is made of.

### SUMMARY

In one aspect the disclosure relates to a medicament container for a liquid medicament. The medicament container comprises a barrel defining a longitudinal direction. The barrel comprises an interior volume. The barrel comprises a hollow space defining or forming the interior volume. The interior volume is configured to accommodate the liquid medicament. The barrel of the medicament container comprises a distal end and a proximal end. The barrel comprises an outlet for the liquid medicament at the distal end. Typically, the distal end is or represents one longitudinal end of the barrel. The proximal end forms or constitutes the oppositely located longitudinal end of the barrel.

The barrel may provide a primary packaging for the liquid medicament. Hence, the barrel may get in direct contact with the liquid medicament and may confine the interior volume. The interior volume is configured to become completely occupied by the liquid medicament. Typically, the material of the barrel is chemically and/or pharmaceutically inert with regards to the liquid medicament. The barrel may therefore provide long-term storage of the liquid medicament therein.

The outlet forms a distal end of the interior volume. The interior volume is confined in the lateral direction, i.e. perpendicular to the longitudinal direction, by the barrel, in particular by a sidewall of the barrel.

The medicament container further comprises a mount for an electronic identifier. The medicament container is further provided with an electronic identifier. The electronic identifier is engaged with the mount. The electronic identifier is attached to the barrel by the mount.

The mount provides a manual mutual assembly of the electronic identifier and the barrel. With some examples the mount is non-detachably or non-releasably arranged and fixed to the barrel. The mount may be integrated into the barrel or the mount may be implemented as an integral part or portion of the barrel. With other examples the mount may be detachable from the barrel. Here, the mount may comprise a fastening member to engage with the barrel or to engage with a counter fastening member of the barrel.

The mount is particularly configured and shaped with respect to the geometric dimensions of the electronic identifier. In this way, the mount provides a mechanical fixing structure for the electronic identifier and thus enables a mechanical and/or manual fixing of the electronic identifier to the barrel.

The medicament container optionally comprises a closure sealing the barrel towards the proximal end. The closure may be movably arranged inside the barrel. The closure seals the barrel towards the proximal end. With some examples, the closure comprises a piston movably arranged inside the barrel and sealing the barrel towards the proximal end.

The closure or piston may be arranged at or near the proximal end of the barrel. The closure or piston may be also arranged at a predefined distance from the proximal end. However, the closure or piston and the outlet confine the interior volume of the barrel with regards to the longitudinal direction.

With some examples the mount provides a undetachable fastening of the electronic identifier to the mount. An undetachable fastening means, that once the electronic identifier has been attached to the barrel and/or to the mount it cannot be detached from the barrel or from the mount without destroying at least one of the barrel, the mount or the electronic identifier. The undetachable and/or non-releasable attachment of the electronic identifier to the mount prevents an inadvertent disassembly of the electronic identifier from the barrel. In this way and once the electronic identifier has been appropriately assembled to the mount it serves to persistently identify the barrel and/or the medicament located therein.

According to a further example the mount comprises a receptacle. The receptacle comprises an insert opening sized to receive the electronic identifier. Of course, the receptacle is also sized to receive the electronic identifier therein. The electronic identifier can be easily inserted into the receptacle via the insert opening. Typically, the insert opening and/or the receptacle is or are sized to receive the electronic identifier therein in a press-fit. In this way, the electronic identifier may be easily inserted or urged into the receptacle and/or through the insert opening into a final assembly configuration, in which the electronic identifier in its entirety is located inside the receptacle.

When in the final assembly configuration or assembly position inside the receptacle the electronic identifier may no longer protrude from or through the insert opening. Hence, when appropriately assembled in the receptacle and when appropriately assembled to the mount the electronic identifier is no longer mechanically accessible from outside the receptacle and/or from outside the insert opening. Pulling out or unplugging of the electronic identifier from or out of the receptacle is therefore effectively prevented.

According to another example the mount comprises at least one retention feature. The at least one retention feature is configured to fix the electronic identifier in the receptacle. The retention feature is at least one of resiliently deformable or pivotally supported on or in the receptacle. The retention feature serves to prevent a pulling out or unplugging of the electronic identifier from the receptacle. The retention feature may be biased by a resilient member, such as a spring element. The retention feature may effectively close or cover the receptacle towards the insert opening. Typically, the at least one retention feature is arranged at a distance from a dead end of the receptacle such that the electronic identifier can be placed between the dead end of the receptacle and the at least one retention feature.

With some examples the at least one retention feature is configured to protrude into at least one of the receptacle and the insert opening of the receptacle. In this way and e.g. in an initial or unbiased state, the at least one retention feature serves to reduce the cross-section of at least one of the receptacle and the insert opening.

Upon insertion of the electronic identifier into the receptacle the at least one retention feature may be pivoted or resiliently deformed outwardly, and hence in such a way that the cross-section of the receptacle and/or the cross-section of the insert opening is at least temporally enlarged to such a degree, that the electronic identifier is allowed to pass through. When the electronic identifier has reached a final assembly position or final assembly configuration the electronic identifier has typically passed the at least one retention feature, which may then return into the unbiased state, in which the receptacle and/or the insert opening features a reduced cross section.

In the unbiased or in the initial state the at least one retention feature provides an end stop for the electronic identifier. Here, the electronic identifier may be in mechanical engagement and/or in mechanical abutment with the at least one retention feature. Here, the at least one retention feature provides a mechanical fixing and abutment with regards to a pulling out movement. A dead end of the receptacle may provide a corresponding stop or abutment surface with regards to an insert direction.

According to a further example the at least one retention feature comprises at least one catch element. The catch element is configured to engage with the electronic identifier when the electronic identifier is arranged in a predefined assembly position inside the receptacle. Here, the predefined assembly position may coincide with the above-mentioned final assembly position of final assembly configuration. The at least one catch element is one example of the at least one retention feature. The at least one catch element may comprise a beveled section facing towards the insert opening when the catch element is arranged inside the receptacle, e.g. at a sidewall of the receptacle. In this way the at least one catch element and hence the retention feature may become subject to a laterally directed evasive movement when the electronic identifier is urged into the receptacle along an insert direction.

According to another example the electronic identifier comprises at least one of a rigid substrate and a rigid housing. The at least one of the rigid substrate and the rigid housing is mechanically engageable with the mount. The rigid substrate and/or the rigid housing is made of a rather rigid material, i.e. a material that is effectively incompressible. The rigid substrate may comprise a thermoplastic material or a polymeric material as well as a metal material and/or combinations thereof. The rigid substrate and/or the rigid housing is or are configured to mechanically engage with the mount. Moreover, at least one of the rigid substrate and the rigid housing is or are configured to squeeze into the receptacle and/or into the insert opening.

The rigid substrate and/or the rigid housing are particularly configured to engage with the retention feature. The rigid substrate and/or the rigid housing provide a well-defined and robust engagement with the retention feature and/or with the catch element of the retention feature. In this way a rather uncontrolled or unintended release of the electronic identifier from the catch element, the retention feature, the receptacle and/or from the insert opening is effectively prevented or can be effectively avoided.

According to another example at least one of the rigid substrate and the rigid housing is mechanically clamped and/or mechanically squeezed in the mount or at the mount. Moreover, at least one of the rigid substrate and the rigid housing is mechanically clamped and/or mechanically squeezed in the receptacle of the mount. At least one of the rigid substrate and the rigid housing is mechanically clamped and/or mechanically squeezed in the insert opening of the receptacle. With further examples at least one of the rigid substrate and the rigid housing is mechanically clamped and/or mechanically squeezed inside the receptacle. Here, the at least one of the rigid substrate and the rigid housing may be mechanically squeezed and/or mechanically clamped between a dead end or a bottom of the receptacle and the at least one retention feature.

The mechanically clamped or mechanically squeezed arrangement of the at least one of the rigid substrate and the rigid housing of the electronic identifier provides a slack-free and/or tolerance compensating assembly of the electronic identifier inside the receptacle and hence to the mount. The mechanically clamped and/or mechanically squeezed arrangement is easy to manufacture and is also intuitively understandable by an operator, a user or an assembly worker. At the same time, the mechanically clamped and/or mechanically squeezed arrangement of at least one of the rigid substrate and the rigid housing of the electronic identifier provides a robust, durable and reliable mechanical fixing of the electronic identifier to the barrel of the medicament container.

In another example the electronic identifier comprises a transparent substrate. With further examples the electronic identifier comprises a transparent housing. The transparent substrate and/or the transparent housing may be a rigid substrate and/or a rigid housing. With some examples the transparent substrate is a flexible transparent substrate. With some examples the transparent housing is a transparent and flexible housing.

A transparent substrate and/or a transparent housing enables transmission of electromagnetic radiation through the respective substrate or housing. In this way, the electronic identifier can be arranged in a region of the barrel that is substantially transparent. Moreover, the electronic identifier may be arranged at a transparent or semitransparent sidewall portion of the barrel, which sidewall portion is intended for visual inspection of the content of the barrel. Arranging the transparent substrate to the respective transparent barrel portion or sidewall portion does not alter or deteriorate a visual inspection capability of the content of the barrel.

With further examples the transparent substrate may comprise a visual scale, which when attached to the barrel of the medicament container may indicate the filling level of the medicament container. The visual scale may be provided on the transparent substrate. With other examples the visual scale is directly provided or imprinted on a transparent portion of the barrel, e.g. on a transparent sidewall portion of the barrel.

With some examples, the entire barrel is made of a transparent material. The transparent material of the barrel may be semitransparent. It may be dye-doped and/or colored.

According to another example the electronic identifier is a wireless electronic identifier. The electronic identifier comprises an antenna and a processing unit. The processing unit is electrically connected to the antenna. The processing unit is configured to communicate with at least one external electronic device via the antenna. With some examples the electronic identifier is a radiofrequency identification identifier (RFID) configured to operate in a radiofrequency electromagnetic range. Hence, the electronic identifier may be implemented as a RFID tag. The electronic identifier may be configured to operate in at least one of the LF band or HF band. The electronic identifier may be implemented as a passive or as an active RFID tag. The electronic identifier may be implemented as a NFC tag, hence a new field communication tag. With other examples the electronic identifier may be configured to communicate with an external electronic device according to the Bluetooth communication standard or in accordance to a Wi-Fi standard, (IEEE 802.11).

With some examples the electronic identifier comprises an electronic storage electrically connected to the processing unit. The electronic storage may be implemented as a read only storage or as a read-write storage.

When implemented as a read only electronic storage, the electronic identifier may communicate with an external electronic device and may provide only data that has been previously stored in the electronic storage before the electronic identifier has been engaged with the mount or has been assembled inside the receptacle of the barrel. When the electronic storage is implemented as a read only storage, the data stored with the electronic storage may comprise at least one of: a manufacturing day, a manufacturing time, a best before date, a manufacturing lot, an individual manufacturing ID, a manufacturer information such as a manufacturer ID, a manufacturing location.

With other examples and when the electronic storage is implemented as a combined read-write storage the electronic storage may provide a predefined storage capacity for storing medicament container-relevant information during manufacturing, during assembly, during shipment, during storage, during retail and/or during use of the medicament container. The processing unit of the electronic identifier may be configured to retrieve and to process data from an external electronic device and to write the respective data in the electronic storage. Upon request and/or at the end of life cycle of the medicament container and/or of the electronic identifier respective information or data stored in the electronic storage may be retrieved and collected by the external electronic device.

The data stored and/or written in the electronic storage after the electronic identifier has been assembled to the amount of the medicament container may comprise data such as: date of packaging, date of shipment, date of retail, date of first use, data of final use, environmental data, such as temperature, humidity, administration data, such as the amount of the medicament dispensed or expelled at a given point of time or day. Of course, when the electronic storage is implemented as a read-write electronic storage the data types as described before in connection with a read only electronic storage may be also present and stored in the read-write electronic storage.

According to another example the barrel comprises a mechanical connection interface. The mechanical connection interface is configured to connect the barrel to a body of an injection device. The mechanical connection interface comprises at least one fastening feature configured to mechanically engage with a complementary-shaped counter fastening feature of the body of the injection device. The mechanical connection interface of the barrel may engage with a complementary-shaped counter connection interface of the body of the injection device.

At least one of the mechanical connection interface and the mechanical counter connection interface is provided with a mechanical coding such that only a mechanical connection interface of a type A is mechanically engageable with a particular type A body or injection device comprising a complementary or correspondingly-shaped mechanical counter connection interface.

In this way it is guaranteed, that only such medicament containers can be mechanically connected and/or mechanically engaged with a body of such drug delivery devices or injection devices that are intended for use with that particular medicament container. Hence, a medicament container of type A can be only connected with a body of type A. A medicament container of type B cannot be connected with a body of type A. Vice versa, a medicament container of type B can be only connected to a body of type B but not to a body of type A.

Undesirable or prohibited cross use of medicament containers not intended for a particular injection device cannot be used with such non-intended injection devices. The mechanical coding or mechanical encoding of the mechanical connection interface and the mechanical counter connection interface may be integrated into the fastening feature and counter fastening feature, respectively.

The mechanical connection feature and the correspondingly shaped mechanical counter connection feature may comprise at least one snap feature configured to engage with a complementary-shaped counter snap feature or with a complementary shaped recess. The snap feature may be provided on one of the mechanical connection features and the mechanical counter connection features. The counter snap feature will then be provided on the other one of the mechanical connection feature and the mechanical counter connection feature.

With some examples the fastening feature and the counter fastening feature form a threaded connection or a bayonet connection. Also here, the mutually engaging components, e.g. radial protrusions and recesses are configured to engage only when a respective mechanical coding of the mechanical connection interface matches with the mechanical coding of the correspondingly or complementary shaped mechanical counter connection interface.

With some examples, the medicament container is directly attachable to a proximal housing component of the drug delivery device. The drug delivery device may be implemented as an injection device. The injection device may comprise or may be implemented as a pen-type injector. With a direct mechanical engagement between the above-described medicament container and a body of a housing of an injection device the housing of the injection device may be void of a separate cartridge holder configured to receive and to mechanically fix a product or medicament containing cartridge. Rather, the medicament container as described above may be implemented as a combined cartridge holder and cartridge.

With some examples the medicament container comprises or constitutes a medicament cartridge having a barrel substantially filled with the liquid medicament. The barrel may comprise a mechanical interface which is directly connectable to a componentry shaped counter interface of the housing of the drug delivery device. The drug delivery device may be implemented as a handheld injection pen. With these examples the injection needle may be provided with a needle hub configured for detachable and releasable connection to the outlet of the barrel. The distal outlet end of the barrel may be sealed by a pierceable seal, e.g. implemented as a sealing disc, such as a septum. The seal may be mechanically fixed to the outlet of the barrel by means of a beaded cap, e.g. made of a plastically deformable metal. With some examples the barrel does not only provide an interior volume to directly accommodate the liquid medicament. The barrel also comprises a proximally located mechanical connection interface for releasably or non-releasably fastening the barrel and hence the medicament container to the body of the injection device. Typically, the body of the injection device accommodates or holds a drive mechanism configured to engage with the medicament container for expelling or withdrawing the liquid medicament from the interior volume of the container. With such examples, wherein the container comprises a piston movably arranged inside the barrel the drive mechanism comprises a driver or a piston rod configured to engage with the piston of the medicament container in order to displace or to move the piston in distal direction relative to the barrel of the medicament container.

With some further examples the outlet for the liquid medicament is provided with a fastening structure for a fluid guiding structure, such as an injection needle or an infusion line. Here, the outlet may be provided with a standardized mechanical interface configured to detachably or to releasably engage with at least one of a connector of a needle assembly or with a connector of an infusion line. For instance, the outlet of the barrel may be provided with a threaded structure or section. The threaded section may be implemented as an outer threaded section configured to mate with an inner thread of a connector of a needle assembly, e.g. a needle hub.

With some other embodiments, the threaded section of the barrel may be implemented as an inner threaded section configured to mate with an outer thread of a connector of a needle assembly, e.g. of a needle hub.

When appropriately attached to the outlet the needle assembly may penetrate the outlet of the medicament container. Here, the outlet may be provided with a pierceable septum sealing the distal end of the barrel and being penetrable by a tipped cannula or the like piercing assembly.

With other examples, the injection needle is permanently and hence non-detachably connected to the outlet of the barrel. It may be molded in the outlet and may thus extend through the outlet.

With some examples, the outlet of the medicament container may be permanently provided with a tipped cannula or injection needle. Here, a tipped cannula or injection needle may be embedded in the outlet of the medicament container. The medicament container may comprise at least one of a narrowing shoulder portion, a spout or nozzle, through which the injection needle may extend. With some examples the injection needle is molded, e.g. insert molded to the outlet or in the outlet and may extend through the outlet. When molded or embedded to or in the outlet of the injection needle is non-detachably connected to the barrel of the medicament container. A proximal end of the injection needle may be located inside the interior volume of the barrel whereas a distal end of the injection needle may be located outside the barrel and may thus protrude from the distal end of the barrel or from the outlet of the barrel.

With some other examples, the outlet of the medicament container comprises a container interface configured to releasably engage with a needle interface. The container interface and the needle interface may provide at least one of a friction fit and a form fit. with some examples, the container interface comprises a threaded structure correspondingly or complementary shaped to a counter threaded structure of the injection needle. The counter threaded structure of the injection needle is typically provided on a needle hub. It may be implemented as an outer threaded portion or as an inner threaded portion to engage with a correspondingly shaped threaded structure of the container interface.

With some examples, the container interface and the needle interface comprising a bayonet type coupling. Here, at least one of the container interface and the needle interface comprises an elongated groove or slot, comprising an L-shaped geometry. The other 1 of the container interface and the needle interface comprises at least one protrusion to engage with the elongated groove or slot.

With some examples, the container interface and the needle interface may comprise mutually corresponding standardized connectors, such as Luer-type connectors.

With some examples, the medicament container is provided as a syringe or may constitute a syringe. When implemented as a syringe, the medicament container is typically designed and intended for a one-time use only. The medicament container, hence the barrel thereof may be prefilled with the liquid medicament.

According to another example the mount is arranged on an outside surface of the barrel. In this way, the mount is easily accessible from outside the barrel. During manufacturing and/or in a final step of assembly of the medicament container the electronic identifier may be easily insertable into the receptacle or may be easily attachable to the mount provided on the outside of the barrel. This particular arrangement provides a rather intuitive and easy mutual assembly of the electronic identifier and the mount.

According to a further example the electronic identifier is fixed to the mount by the body of the injection device or drug delivery device when the at least one fastening feature is engaged with the counter fastening feature. Here, the mutual mechanical interconnection between the medicament container and the body provides synergy to the fixing of the electronic identifier to the mount. The mount may be arranged in such a way on the outside of the barrel and in a well-defined distance or well-defined orientation or configuration with regard to the at least one fastening feature that upon engagement between the fastening feature and the counter fastening feature the body engages with the electronic identifier thereby retaining and fixing the electronic identifier to the mount, e.g. inside the receptacle.

With some further examples the insert opening of the receptacle of the mount is covered or closed by a component of the injection device, wherein the at least one fastening feature of the medicament container is engaged with the counter fastening feature of the body. With some examples the component of the injection device covering or closing the insert opening is a portion of the boy of the injection device. In this way and when reaching a final assembly configuration between the medicament container or barrel with the body of the injection device the receptacle and/or the mount for the electronic identifier is effectively closed or covered by the component of the injection device and/or by the body. As long as the body is mechanically fixed to the barrel of the medicament container the electronic identifier located inside the receptacle is and remains inaccessible.

With some examples the barrel is non-detachably connectable or is non-detachably connected to the body of the injection device. The barrel and the body may be adhesively interconnected. The barrel of the medicament container and the body of the drug delivery device may be mutually welded. With some other examples the body and the barrel may be non-releasably attached by a snap-fit engagement. Release or disassembly of the snap-fit engagement between the barrel and the body may be only obtained by destroying or disintegrating at least one of the barrel and the body. A nondestructive disassembly or disconnection of the body and the barrel may be prevented by the type and/or by the configuration of the mutually corresponding fastening feature and counter fastening feature.

According to a further example the barrel comprises an elongated structure defining a longitudinal direction and a radial direction. Typically, the radial direction extends perpendicular to the longitudinal direction. The insert opening of the receptacle is open towards the longitudinal direction. Alternatively, the insert opening of the receptacle is open towards the radial direction. With both implementations, covering of the insert opening by at least a portion of the body of the injection device is possible and within the presently disclosed solution.

For the mutual assembly of the barrel and of the body of the injection device at least one of a proximal end of the barrel at a distal end of the body comprises a receptacle configured to receive an insert portion provided on the other one of the proximal end of the barrel and the distal end of the body. In this way, a nested or interleaved mutual assembly configuration of the barrel and of the body can be obtained. With some examples, the barrel comprises a radially stepped down insert portion at the proximal end configured to be received by a receptacle provided at a distal end of the body of the injection device.

With these examples the mount may be either arranged longitudinally adjacent to at least one of the receptacle and the insert. It may be arranged adjacent to an inside section of e.g. the barrel or the insert opening and may face in a proximal direction. Upon reaching a final assembly configuration of the barrel to the body a distal end face of the body may get into abutment with the stepped down portion of a distal end of the insert section of the barrel. In this way the distal end face of the receptacle of the body may at least partially cover or may entirely cover the insert opening of the receptacle in which receptacle the electronic identifier is located or arranged.

With another example and when the barrel comprises a receptacle at the proximal end configured to receive an insert section provided at the distal end of the body the mount for the electronic identifier may be located on an inside facing sidewall portion of the barrel longitudinally adjacent to the insert section-receiving receptacle of the barrel. Here, the receptacle for the electronic identifier may be located at an inside sidewall portion of the barrel and may thus protrude radially inwardly from the inside facing sidewall of the barrel. The insert section of the body may equally serve to cover or close the insert opening of the electronic identifier receptacle. Here, the distal end face of the body may provide a proximal end stop for the electronic identifier arranged inside the receptacle of the mount.

With other examples the mount for the electronic identifier is located on in or on a sidewall of the insert section and the insert section receiving receptacle of one of the barrel and the body of the injection device. The receptacle may be configured to entirely receive the electronic identifier. Here, the receptacle is open towards the radial outer direction or radial inner direction. For instance and when the mount for the electronic identifier is located on or integrated into the sidewall of the barrel it may be provided on an outside facing sidewall of the insert section of the barrel. When reaching a final assembly configuration the insert section of the barrel is covered by an inside facing sidewall of the insert-receiving receptacle of the body of the injection device. Then, the insert opening of the electronic identifier receptacle is closed and/or covered in radial direction by the sidewall of the body, e.g. by a sidewall of the receptacle of the body.

With another example, wherein the barrel comprises a body-receiving receptacle at a proximal end the respective mount and hence the receptacle for the electronic identifier is arranged and/or integrated into an inside facing sidewall portion of the insert section-receiving receptacle of the barrel. Here and when reaching a final assembly configuration of the barrel and the body the insert section of the body covers and/or closes the electronic identifier receiving receptacle provided on the inside facing sidewall of the receptacle of the barrel.

According to a further example the barrel is made of a plastic material. The plastic material may comprise a material composition. The plastic material and/or the material composition may comprise any of cyclic olefin copolymer (COC) and/or cyclo-olefin polymer (COP). The plastic material and/or the composition materials can be configured to have a high double refraction (e.g., optical refractive index higher than 1.5), to provide a high moisture barrier (e.g., moisture absorption smaller than 0.01) and a good material strength (e.g., a charpy impact strength of about 13 to 15). For example, plastic materials, such as COC materials include high purity, high moisture barrier, excellent double refraction, breakage prevention, and low density. Most COC grades can undergo sterilization by gamma radiation, by high temperature steam, or by ethylene oxide. COC also has a very low energy and a nonreactive surface, which can extend shelf life and purity of medications, such as insulin and other protein drugs, stored in medicament containers.

With some examples the mount is integrally formed with the barrel or the mount is integrated into the barrel. This applies particularly, when the barrel is made of a plastic material.

The barrel of the medicament container as such and/or the entire medicament container may be made of such a plastic or polymeric material. The barrel and/or the entire medicament container may be manufactured and/or provided as an injection molded plastic component. The medicament container may comprise a single-piece component. Hence, the medicament container and the barrel, the mount and/or the insert opening as well as the retention features thereof may be integrally formed. The numerous components or parts of the medicament container may be produced in a single injection molding process. This provides a rather fast and cost-efficient manufacturing of the medicament container. For a final assembly of the medicament container it may be only necessary to seal the distal outlet and to seal the barrel of the container in proximal direction, e.g. with a proximal closure, such as a piston, typically made of an elastomeric or rubber-material.

Moreover and upon final assembly of the medicament container the electronic identifier has to be manually engaged with the mount. Here, the electronic identifier may be simply inserted into the receptacle of the mount. The electronic identifier may be fixed to the mount by the at least one retention feature or the at least one catch element. Alternatively and/or additionally, the electronic identifier may be fixed and/or clamped in the mount or receptacle by engaging and/or connecting the medicament container with the body of the drug delivery device or injection device. The electronic identifier, e.g. a housing or a rigid substrate thereof may be press fitted in the receptacle and/or in the insert opening of the mount.

According to a further the medicament container comprises a syringe and wherein the barrel is a syringe barrel. The barrel may be prefilled with the liquid medicament. The barrel may be of tubular shape. It comprises a distal end provided with the outlet and further comprises a proximal end. The proximal end of the barrel is typically provided with a finger flange allowing to hold the barrel or the syringe with the index finger and the middle finger of a hand of a user.

The outlet of the barrel or of the syringe may comprises a standardized container interface to releasably and/or to detachably engage with a complementary-shaped needle interface. In this way, the injection needle or a respective needle hub thereof can be detachably connected to the outlet of the syringe. With other examples, the outlet of the barrel or of the syringe is permanently and non-detachably connected with the injection needle. Here, the injection needle may be molded to the outlet and may extend through the outlet.

With some examples the outlet of the barrel comprises a stepped down neck portion or a shoulder portion. Here, the barrel may extend into a spout are nozzle. The injection needle may extend through the spout or nozzle or may be connectable to such spout or nozzle. The container interface may be provided in or on the spout or nozzle provided at the outlet of the barrel or syringe.

The syringe, hence its barrel may be directly filled with the liquid medicament. Towards a proximal end, the interior volume confined by the barrel may be sealed by a piston movably arranged inside the barrel. The piston is sealingly engaged with an inside surface of the sidewall of the barrel. The piston is further provided with a longitudinally extending plunger rod. A proximal end of the plunger rod facing away from the piston may be provided with a radially widened flange portion. The flange portion is configured and shaped to receive a dispensing pressure, e.g. exerted by a thumb of a user.

With some examples, the medicament container is filled with a liquid medicament or at least with a lyophilized pharmaceutically active substance. Hence, the medicament container is a prefilled medicament container.

According to another aspect the disclosure relates to a drug delivery device for administering a dose of a liquid medicament. The drug delivery device comprises a housing and a drive mechanism. The housing of the drug delivery device comprises a body and further comprises a medicament container as described above. The drive mechanism is arranged or fixed inside the body and comprises a driver. The driver is operable to engage with the medicament container for expelling or withdrawing the liquid medicament from the medicament container, typically through the distally located outlet of the medicament container.

The drug delivery device may be implemented as an injection device or as an inhaler. The drug delivery device is typically implemented as one of a single shot device, as a multiple shot device, as a disposable device, as a reusable device, as a fixed dose device or as a variable dose device.

The drive mechanism and/or the driver thereof may comprise a piston rod movable in longitudinal direction and operable to move a piston of the medicament container in distal direction in order to expel or to withdraw the liquid medicament from the medicament container, e.g. through the distally located outlet thereof.

The injection device may be implemented as a reusable device or as a disposable device. With a disposable device the medicament container is prefilled with a liquid medicament and the medicament container may be undetachably fixed to the body of the housing of the injection device. Also here, the medicament container may comprise or may constitute a syringe intended for a one-time injection. When the content of the medicament container has been used up or when the content should not be used any longer, e.g. because a best before date has exceeded or reached the entirety of the injection device or syringe is intended to be discarded.

With a reusable drug delivery device, such as an injection device, the medicament container as described above may be detachably and hence reversibly connected to the body of the injection device or drug delivery device. Once the content of the medicament container has been used up or once the content should not be used any longer the entire medicament container is replaced by a new one. For this, the medicament container is detached from the body and another medicament container with a suitable fastening feature is attached to the body.

According to another aspect the injection device comprises a medicament container, wherein the medicament container is filled with a liquid medicament. The liquid medicament may be or may comprise an injectable medicament. The liquid medicament may be provided in a liquid or powdered constitution inside the medicament container. The liquid medicament may be provided in powdered and/or lyophilized form inside the medicament container. For preparing the liquid medicament from a respective powder adding of a solvent or diluent to the interior of the medicament container may be required. The diluent or solvent may be added to the interior volume of the barrel through the outlet of the barrel. For this, the outlet of the barrel may be penetrated with a suitable cannula.

Generally, the scope of the present disclosure is defined by the content of the claims. The injection device is not limited to specific embodiments or examples but comprises any combination of elements of different embodiments or examples. Insofar, the present disclosure covers any combination of claims and any technically feasible combination of the features disclosed in connection with different examples or embodiments.

In another aspect the present disclosure relates to a secondary medicament container for a liquid medicament. In the present context, a secondary medicament container represents or defines a type of a medicament container that does not get in direct contact with the liquid medicament. Rather, the secondary medicament container is intended and configured to receive or to accommodate a primary medicament container. The primary medicament container is intended and configured to directly receive the liquid medicament. Hence, at least a portion of the primary medicament container is in direct contact with the liquid medicament when e.g. prefilled with the liquid medicament.

Hence, the secondary medicament container is implemented and/or configured to receive or to accommodate a primary medicament container, wherein the primary medicament container is configured to hold or to accommodate the liquid medicament directly therein. In other words, the medicament may be accommodated or located inside the primary medicament container and the primary medicament container with the medicament located therein can be located in and/or accommodated by the secondary medicament container.

The secondary medicament container comprises a barrel defining a longitudinal direction and comprising an interior volume to accommodate the primary medicament container. The barrel of the secondary medicament container comprises a distal end and a proximal end. The barrel comprises an outlet for the liquid medicament at the distal end.

The secondary medicament container comprises a mount for an electronic identifier. The medicament container further comprises an electronic identifier engaged with the mount and attached to the barrel of the secondary medicament container via the mount.

Typically, the secondary medicament container is configured to receive or to accommodate a primary medicament container as described above with the exception, that the primary medicament container may be void of a mount for an electronic identifier and that the primary medicament container may be void of such an electronic identifier. Instead, the mount for the electronic identifier as described above in connection with the primary medicament container is now provided with the secondary medicament container. Hence, the electronic identifier is engaged with the barrel of the secondary medicament container via the mount of the second medicament container.

With some examples, the secondary medicament container is implemented as a cartridge holder of a drug delivery device, e.g. as a cartridge holder of an injection device, such as a pen-type injector. The secondary medicament container may be detachably connected to a body of the drug delivery device or injection device. With some examples the secondary medicament container may be non-detachably but permanently connected to the body of the drug delivery device.

The secondary medicament container may be implemented as a component of a disposable drug delivery device, such as a disposable injection device. Such disposable injection devices are intended to be discarded after the liquid medicament, hence the content of the primary medicament container, has been used up. In particular and when the injection device is intended to be discarded after use consumption of the medicament located inside the medicament container the electronic identifier can be attached to any of the barrel of the primary medicament container or of the secondary medicament container.

With some examples, the proximal end of the secondary medicament container is provided with a mechanical interface configured to engage with a correspondingly-shaped mechanical counter interface of a drug delivery device. The mechanical interface of the proximal end of the secondary medicament container typically provided with a respective fastening feature. This allows to releasably or non-detachably connect the secondary medicament container to the housing or body of a drug delivery device.

With some examples the distal end of the secondary medicament container comprises an outlet. The outlet may comprise a through opening, through which an outlet of the primary medicament container extends or through which outlet a needle assembly may extend to engage with the outlet of the primary medicament container. With some examples the distal end of the secondary medicament container comprises a container interface complementarity shapes to a needle interface of a needle assembly. With some examples, the container interface of the secondary medicament container comprises an outer thread to engage with an inner thread of the needle assembly. With other examples, the container interface of the secondary medicament container comprises an inner thread to engage with a correspondingly shaped outer thread of the needle assembly.

When intended for use with a tipped injection needle, the outlet of the secondary medicament container is particularly configured to receive a proximal end of the injection needle therethrough. Here, the needle hub of the needle assembly may mechanically engage with the distal end of the secondary medicament container whereas the proximal end of the double-tipped injection needle extends through the outlet end of the secondary medicament container and through an outlet of the primary medicament container.

At least a portion of the secondary medicament container, e.g. a portion of a sidewall of the barrel of the secondary medicament container is transparent or comprises a through opening through which the interior volume of the secondary medicament container can be visually inspected. This allows for a visual inspection of the medicament located inside the first and secondary medicament containers. Moreover, a filling level of the primary medicament container can be visually inspected from outside the secondary medicament container.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The term “drug” or “medicament”, as used herein, means a pharmaceutical formulation containing at least one pharmaceutically active compound,

wherein in one embodiment the pharmaceutically active compound has a molecular weight up to 1500 Da and/or is a peptide, a protein, a polysaccharide, a vaccine, a DNA, a RNA, an enzyme, an antibody or a fragment thereof, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exendin-3 or exendin-4 or an analogue or derivative of exendin-3 or exendin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-Y-glutamyl)-des(B30) human insulin; B29-N—(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser- Ser-Gly-Ala-Pro-Pro-Pro-Ser-NH2.

Exendin-4 derivatives are for example selected from the following list of compounds:

H-(Lys)4-des Pro36, des Pro37 Exendin-4(1-39)-NH2,

H-(Lys)5-des Pro36, des Pro37 Exendin-4(1-39)-NH2,

des Pro36 Exendin-4(1-39),

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39); or

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39),

wherein the group -Lys6-NH2 may be bound to the C-terminus of the Exendin-4 derivative;

or an Exendin-4 derivative of the sequence

des Pro36 Exendin-4(1-39)-Lys6-NH2 (AVE0010),

H-(Lys)6-des Pro36 [Asp28] Exendin-4(1-39)-Lys6-NH2,

des Asp28 Pro36, Pro37, Pro38Exendin-4(1-39)-NH2,

H-(Lys)6-des Pro36, Pro38 [Asp28] Exendin-4(1-39)-NH2,

H-Asn-(Glu)5des Pro36, Pro37, Pro38 [Asp28] Exendin-4(1-39)-NH2,

des Pro36, Pro37, Pro38 [Asp28] Exendin-4(1-39)-(Lys)6-NH2,

H-(Lys)6-des Pro36, Pro37, Pro38 [Asp28] Exendin-4(1-39)-(Lys)6-NH2,

H-Asn-(Glu)5-des Pro36, Pro37, Pro38 [Asp28] Exendin-4(1-39)-(Lys)6-NH2,

H-(Lys)6-des Pro36 [Trp(O2)25, Asp28] Exendin-4(1-39)-Lys6-NH2,

H-des Asp28 Pro36, Pro37, Pro38 [Trp(O2)25] Exendin-4(1-39)-NH2,

H-(Lys)6-des Pro36, Pro37, Pro38 [Trp(O2)25, Asp28] Exendin-4(1-39)-NH2,

H-Asn-(Glu)5-des Pro36, Pro37, Pro38 [Trp(O2)25, Asp28] Exendin-4(1-39)-NH2,

des Pro36, Pro37, Pro38 [Trp(O2)25, Asp28] Exendin-4(1-39)-(Lys)6-NH2,

H-(Lys)6-des Pro36, Pro37, Pro38 [Trp(O2)25, Asp28] Exendin-4(1-39)-(Lys)6-NH2,

H-Asn-(Glu)5-des Pro36, Pro37, Pro38 [Trp(O2)25, Asp28] Exendin-4(1-39)-(Lys)6-NH2,

H-(Lys)6-des Pro36 [Met(O)14, Asp28] Exendin-4(1-39)-Lys6-NH2,

des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

H-Asn-(Glu)5-des Pro36, Pro37, Pro38 [Met(O)14, Asp28] Exendin-4(1-39)-NH2,

des Pro36, Pro37, Pro38 [Met(O)14, Asp28] Exendin-4(1-39)-(Lys)6-NH2,

H-(Lys)6-des Pro36, Pro37, Pro38 [Met(O)14, Asp28] Exendin-4(1-39)-(Lys)6-NH2,

H-Asn-(Glu)5 des Pro36, Pro37, Pro38 [Met(O)14, Asp28] Exendin-4(1-39)-(Lys)6-NH2,

H-Lys6-des Pro36 [Met(O)14, Trp(O2)25, Asp28] Exendin-4(1-39)-Lys6-NH2,

H-des Asp28 Pro36, Pro37, Pro38 [Met(O)14, Trp(O2)25] Exendin-4(1-39)-NH2,

H-(Lys)6-des Pro36, Pro37, Pro38 [Met(O)14, Asp28] Exendin-4(1-39)-NH2,

H-Asn-(Glu)5-des Pro36, Pro37, Pro38 [Met(O)14, Trp(O2)25, Asp28] Exendin-4(1-39)-NH2,

des Pro36, Pro37, Pro38 [Met(O)14, Trp(O2)25, Asp28] Exendin-4(1-39)-(Lys)6-NH2,

H-(Lys)6-des Pro36, Pro37, Pro38 [Met(O)14, Trp(O2)25, Asp28] Exendin-4(S1-39)-(Lys)6-NH2,

H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exendin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Antibodies are globular plasma proteins (˜150 kDa) that are also known as immunoglobulins which share a basic structure. As they have sugar chains added to amino acid residues, they are glycoproteins. The basic functional unit of each antibody is an immunoglobulin (Ig) monomer (containing only one Ig unit); secreted antibodies can also be dimeric with two Ig units as with IgA, tetrameric with four Ig units like teleost fish IgM, or pentameric with five Ig units, like mammalian IgM.

The Ig monomer is a “Y”-shaped molecule that consists of four polypeptide chains; two identical heavy chains and two identical light chains connected by disulfide bonds between cysteine residues. Each heavy chain is about 440 amino acids long; each light chain is about 220 amino acids long. Heavy and light chains each contain intrachain disulfide bonds which stabilize their folding. Each chain is composed of structural domains called Ig domains. These domains contain about 70-110 amino acids and are classified into different categories (for example, variable or V, and constant or C) according to their size and function. They have a characteristic immunoglobulin fold in which two β sheets create a “sandwich” shape, held together by interactions between conserved cysteines and other charged amino acids.

There are five types of mammalian Ig heavy chain denoted by α, δ, ε, γ, and μ. The type of heavy chain present defines the isotype of antibody; these chains are found in IgA, IgD, IgE, IgG, and IgM antibodies, respectively.

Distinct heavy chains differ in size and composition; α and γ contain approximately 450 amino acids and δ approximately 500 amino acids, while μ and ε have approximately 550 amino acids. Each heavy chain has two regions, the constant region (C_{H}) and the variable region (V_{H}). In one species, the constant region is essentially identical in all antibodies of the same isotype, but differs in antibodies of different isotypes. Heavy chains γ, α and δ have a constant region composed of three tandem Ig domains, and a hinge region for added flexibility; heavy chains μ and ε have a constant region composed of four immunoglobulin domains. The variable region of the heavy chain differs in antibodies produced by different B cells, but is the same for all antibodies produced by a single B cell or B cell clone. The variable region of each heavy chain is approximately 110 amino acids long and is composed of a single Ig domain.

In mammals, there are two types of immunoglobulin light chain denoted by λ and κ. A light chain has two successive domains: one constant domain (CL) and one variable domain (VL). The approximate length of a light chain is 211 to 217 amino acids. Each antibody contains two light chains that are always identical; only one type of light chain, κ or λ, is present per antibody in mammals.

Although the general structure of all antibodies is very similar, the unique property of a given antibody is determined by the variable (V) regions, as detailed above. More specifically, variable loops, three each the light (VL) and three on the heavy (VH) chain, are responsible for binding to the antigen, i.e. for its antigen specificity. These loops are referred to as the Complementarity Determining Regions (CDRs). Because CDRs from both VH and VL domains contribute to the antigen-binding site, it is the combination of the heavy and the light chains, and not either alone, that determines the final antigen specificity.

An “antibody fragment” contains at least one antigen binding fragment as defined above, and exhibits essentially the same function and specificity as the complete antibody of which the fragment is derived from. Limited proteolytic digestion with papain cleaves the Ig prototype into three fragments. Two identical amino terminal fragments, each containing one entire L chain and about half an H chain, are the antigen binding fragments (Fab). The third fragment, similar in size but containing the carboxyl terminal half of both heavy chains with their interchain disulfide bond, is the crystalizable fragment (Fc). The Fc contains carbohydrates, complement-binding, and FcR-binding sites. Limited pepsin digestion yields a single F(ab′)2 fragment containing both Fab pieces and the hinge region, including the H—H interchain disulfide bond. F(ab′)2 is divalent for antigen binding. The disulfide bond of F(ab′)2 may be cleaved in order to obtain Fab′. Moreover, the variable regions of the heavy and light chains can be fused together to form a single chain variable fragment (scFv).

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

It will be further apparent to those skilled in the art that various modifications and variations can be made to the present disclosure without departing from the scope of the disclosure. Further, it is to be noted, that any reference numerals used in the appended claims are not to be construed as limiting the scope of the disclosure.

### BRIEF DESCRIPTION OF THE DRAWINGS

In the following, numerous examples of the injection device comprising a filling level indicator will be described in greater detail by making reference to the drawings, in which:

FIG. 1 schematically illustrates one example of a drug delivery device implemented as an injection device,

FIG. 2 shows an exploded view of a further example of an injection device,

FIG. 3 schematically illustrates one example of an electronic identifier,

FIG. 4 schematically illustrates another example of an electronic identifier,

FIG. 5 schematically illustrates another example of an electronic identifier,

FIG. 6 schematically illustrates another example of an electronic identifier,

FIG. 7 is a perspective illustration of one example of the medicament container equipped with an electronic identifier,

FIG. 8 is another example of the medicament container equipped with an electronic identifier,

FIG. 9 is a cross-section along A-A of FIG. 8,

FIG. 10 schematically illustrated another example of a medicament container equipped with an electronic identifier,

FIG. 11 is a cross-section along B-B of FIG. 10,

FIG. 12 is a longitudinal cross-section through a mechanical interface between the medicament container and a body of the injection device,

FIG. 13 is a block diagram of one example of an electronic identifier,

FIG. 14 shows another example of a medicament container equipped with a mount for an electronic identifier,

FIG. 15 shows another example of a medicament container equipped with a mount for an electronic identifier,

FIG. 16 shows another example of a medicament container equipped with a mount for an electronic identifier,

FIG. 17 is an enlarged and partial view of the mount according to FIGS. 16,

FIG. 18 shows a further example of a medicament container equipped with an electronic identifier,

FIG. 19 shows another embodiment of a medicament container equipped with an electronic identifier as seen from a proximal direction,

FIG. 20 shows the medicament container of FIG. 19 as seen from the distal direction,

FIG. 21 shows another embodiment of a medicament container equipped with an electronic identifier as seen from a proximal direction,

FIG. 22 shows a cross-section through the amount of the medicament container of FIG. 21,

FIG. 23 shows another example of a medicament container as seen from a proximal direction,

FIG. 24 shows a further example of a medicament container as seen from a proximal direction,

FIG. 25 shows a further example of a medicament container, wherein the mount for the electronic identifier is provided near a distal end of its barrel,

FIG. 26 shows another example of a medicament container, wherein the mount for the electronic identifier is provided in a flange of the medicament container,

FIG. 27 shows another example of a syringe type medicament container, wherein the mount for the electronic identifier is provided in a plunger,

FIG. 28 shows another example of a syringe type medicament container, wherein the mount for the electronic identifier is provided in the plunger,

FIG. 29 shows another example of a syringe type medicament container, wherein the mount for the electronic identifier is provided in the plunger and

FIG. 30 shows an example of a secondary medicament container configured to receive a primary medicament container provided with the liquid medicament.

### DETAILED DESCRIPTION

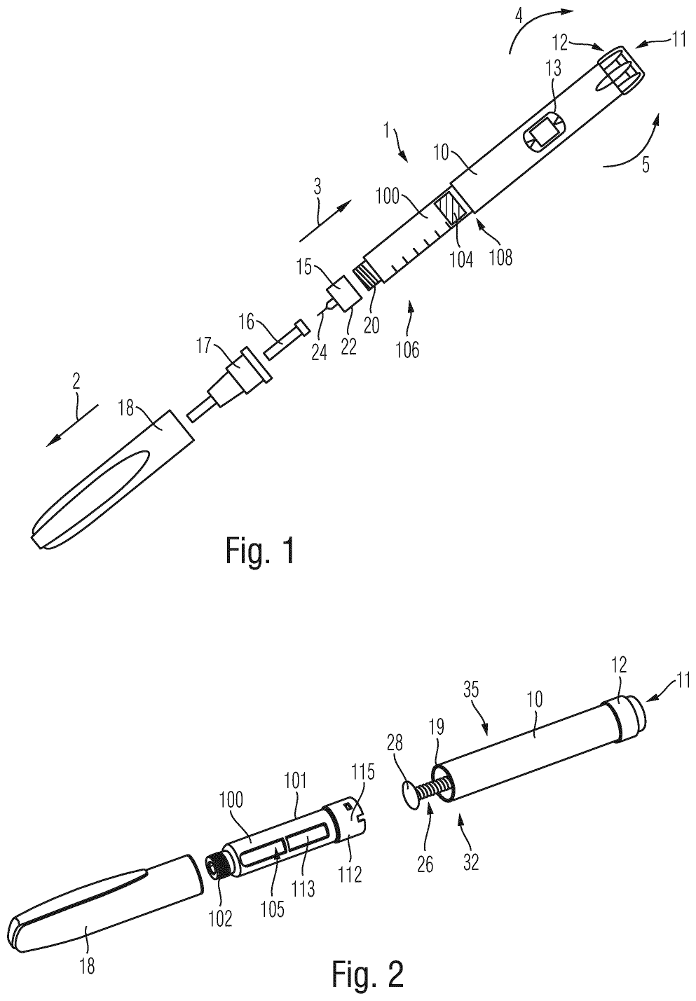

The injection device 1 as shown in FIGS. 1 and 2 can be implemented as a pre-filled disposable injection device that comprises a housing 10 to which a needle hub or needle assembly 15 can be affixed. With some examples the injection device is implemented as a one shot or one-time usable injection device. The injection device may be implemented as a variable dose or as a fixed dose device. When implemented as a fixed dose device the user cannot modify or alter the size of a dose to be dispensed with the injection device. With a variable dose device, a user is given the possibility to set or to modify the size of a dose individually.

An injection needle 24 of the needle assembly 15 is protected by an inner needle cap 16 and either an outer needle cap 17 or a protective cap 18 that is configured to enclose and to protect a distal section of the housing 6 of the injection device 1. The housing 6 may comprise proximally located body 10 configured to accommodate a drive mechanism 30. The injection device 1 further comprises a medicament container 100 the details of which will be described below. The medicament container 100 may be permanently or releasably connected to the body 10. The medicament container 100 is filled with a liquid medicament.

With some embodiments, the injection device 1 is configured and/or implemented as a reusable injection device 1. For such injection devices 1 the medicament container 100 is releasably connectable to the body 10. When the content of the medicament container 100 has been used-up the medicament container 100 can be replaced by another one.

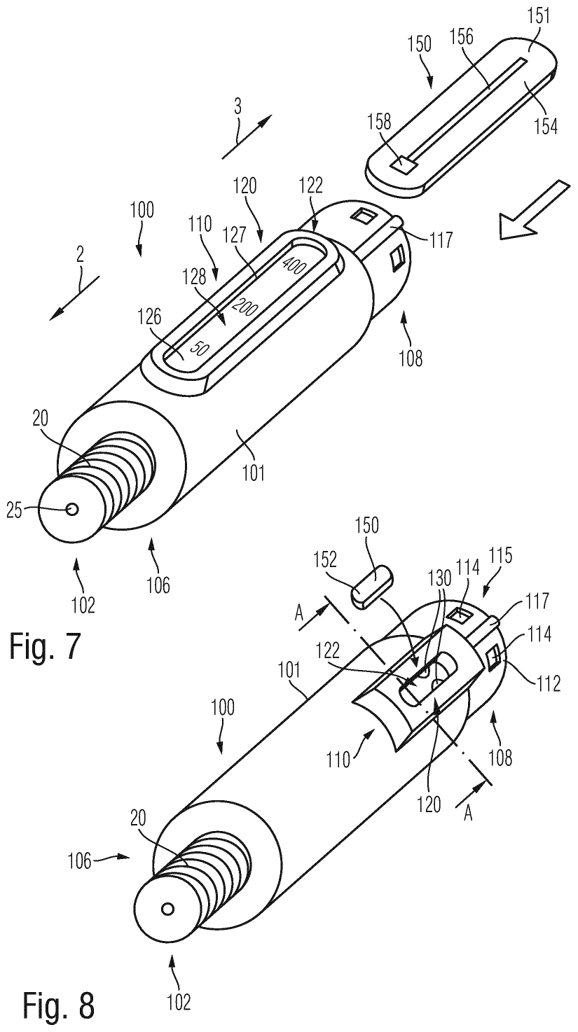

The needle assembly 15 may comprise a threaded needle hub 22 configured for a screwed connection with a threaded section 20 at a distal end 106 of the drug container 100. As illustrated in FIGS. 2 and 7, the very distal end of the medicament container 100 comprises an aperture 25 or a through opening configured to receive a proximally extending tipped end of the injection needle 24. In particular, the injection needle 24 of the needle assembly 15 intersects the needle hub 22 and comprises a double-tipped cannula.

The drive mechanism 30 comprises a driver operable to engage with the medicament container 100 in order to expel or to withdraw the liquid medicament from the medicament container 100. As illustrated, the driver comprises a piston rod 26 operable to urge a piston 104 of the medicament container 100 in distal direction 2 in order to expel the medicament from the medicament container 100. A distal end of the piston rod 26 typically comprises a pressure piece 28 having a radially widened structure compared to the elongated piston rod 26. The pressure piece 28 will get in direct mechanical contact with a proximal face of the piston 104. The piston rod 26 may comprise a threaded rod that is in threaded engagement with the body 10.

The drug delivery device 1 comprises a dose dial 12 and a trigger 11. In the example as illustrated in FIG. 1 the dose dial 12 and the trigger 11 are provided at a proximal end of the housing 10. The injection device 1 further comprises a dosage window 13 in which a size of a dose of the medicament currently set is visually indicated. For setting of a dose the user simply rotates the dose dial 12 in a dose incrementing direction 4 relative to the housing 10. During setting of a dose consecutive increasing numbers will show up in the dosage window 13. If a dose currently set should be too high the user also has the possibility to decrease the size of a dose by dialing or rotating the dose dial 12 in the opposite, hence dose decrementing direction 5. Once a dose of appropriate size has been set or selected a dose dispensing procedure may be triggered or controlled by depressing of the trigger 11, e.g. forming a proximal end face of the injection device. The trigger 11 may be depressed in distal direction 2 by a thumb of a user in order to initiate and/or or to control a dispensing operation of the injection device 1.

The medicament container 100 as for instance illustrated in FIG. 2 comprises a rather tubular-shaped barrel 101 and a distal outlet 102. The distal outlet 102 may be provided with an outer threaded portion 20 configured to engage with a complementary shaped inner threaded section of the needle hub 22. Inside the tubular shaped barrel 101 the elastomeric piston 104 is displaceable in longitudinal direction, e.g. in longitudinal distal direction 2 in order to expel the liquid medicament from the interior volume 105.

At least a portion or the entirety of the barrel 101 is provided with a transparent portion 113 thus allowing to visually inspect the liquid medicament contained inside the medicament container 100. The entirety of the barrel 101 and/or of the medicament container may be made of a transparent plastic material, such as COC, COP or mixtures thereof.

As shown in greater detail in any of the FIGS. 7-18 the medicament container 100 and/or the barrel 101 is provided with a mount 110 for an electronic identifier 150. The mount 110 comprises a receptacle 120 sized to receive the electronic identifier 150. The receptacle 120 typically comprises an insert opening 122 sized to receive the electronic identifier 150. The electronic identifier 150 is non-detachably connectable to the barrel 101 via the mount 110. In other words the electronic identifier 150 is non-detachably connectable to the mount 110 and is non-detachably fixable to the mount 110.

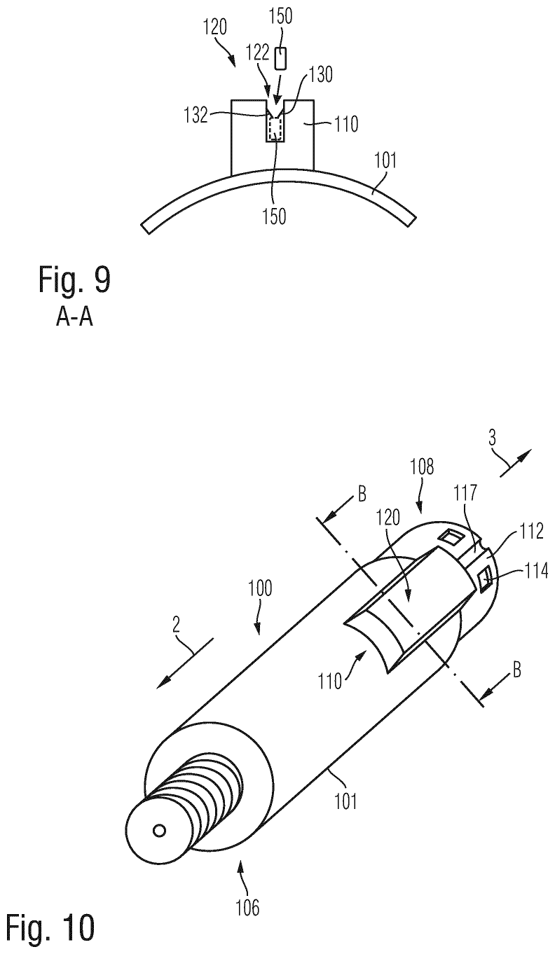

For a non-detachable assembly or fixing of the electronic identifier 150 to the mount 110, the mount 110 comprises at least one retention feature 130 as illustrated in greater detail in FIG. 9. Here, the at least one retention feature 130 is implemented as a catch element 132 extending inwardly from a sidewall of the receptacle 120. The at least one retention feature 130 extends into the inner cross-section of the receptacle 120 and/or of the insert opening 122. The at least one retention feature 130 is at least one of resiliently deformable and/or pivotally supported on or in the receptacle.

The inner cross-section of the receptacle 120 with the retention feature 130 in an initial or unbiased configuration is typically smaller than a respective cross-section of at least one of the electronic identifier 150 or rigid housing 152 thereof. As illustrated in FIG. 9 the electronic identifier 150 can be inserted into the receptacle 120 by pivoting or resiliently deforming the at least one retention feature 130. When arriving in a final assembly position as indicated in FIG. 9 by the dashed lines, the electronic identifier 150 has passed the retention feature 130. Accordingly, the retention feature 130 returns into an initial or unbiased state, in which the retention feature 130 provides an end stop for the electronic identifier 150 thereby preventing a pulling out of the electronic identifier 150 from the receptacle 120 opposite the insert direction.

As illustrated in FIG. 9, there may be provided two or more retention features 130 or two or more catch elements 132 located at opposite sections or portions of the sidewall of the receptacle 120. Once the electronic identifier 150 has reached the final assembly configuration as illustrated in FIG. 9 it cannot be pulled out again without destroying one of the retention features 130, the electronic identifier 150 or the mount 110.

In the example of FIGS. 8 and 9 the barrel 101 of the medicament container 100 is of substantially tubular shape. The mount 110 may protrude radially from an outside surface of the barrel 101. As illustrated in FIG. 8, the mount 110 is located at or near a proximal end 108 of the medicament container 100. In particular, a proximal end of the mount 110 is located adjacent a mechanical interface 112 of the container 100 configured to engage with a mechanical counter interface 32 of the body 10 of the injection device 1. The mechanical interface 112 of the medicament container 100 comprises an insert section 115 at the proximal end 108. The insert section 115 is sized and configured to be received in a correspondingly shaped receptacle 35 at a distal end of the body 10.

For a mutual engagement and for a fixing of the medicament container 100 to the body 10 the medicament container 100 comprises at least one fastening feature 114 configured to engage with a correspondingly shaped or complementary-shaped counter fastening feature 14 provided on the mechanical counter interface 32 of the body 10.

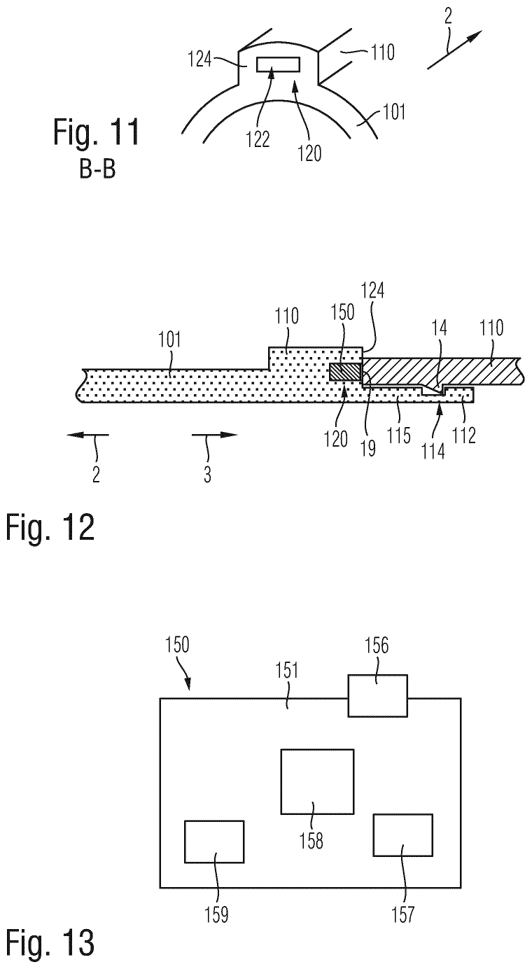

The fastening feature 114 may comprise a recess on the outside surface of the mechanical interface 112 and on the outside of the insert section 115. The counter fastening feature 14 may comprise a correspondingly or complementary shaped protrusion on an inside of the receptacle 35 at a distal end of the body 10, hence in the region of the mechanical counter interface 32 as illustrated in FIG. 12.

The receptacle 120 as illustrated in the examples of FIGS. 8, 9, 15 and 18 is open towards a radial outer direction. Hence, the electronic identifier 150 is insertable into the receptacle 150 along a radially inwardly directed insert direction.

With other examples such as illustrated in FIGS. 7, 10-12, 14, 16 and 17 the receptacle 120 is open towards a longitudinal direction. Hence, the insert opening 122 of the receptacle 120 faces in distal direction 2 or in proximal direction 3.

The mount 110 may be integrally formed with the barrel 101 and hence with the medicament container 100. At least the barrel 101 and the mount 110 may be integrally formed. They may be provided or manufactured by injection molding of the barrel 112.

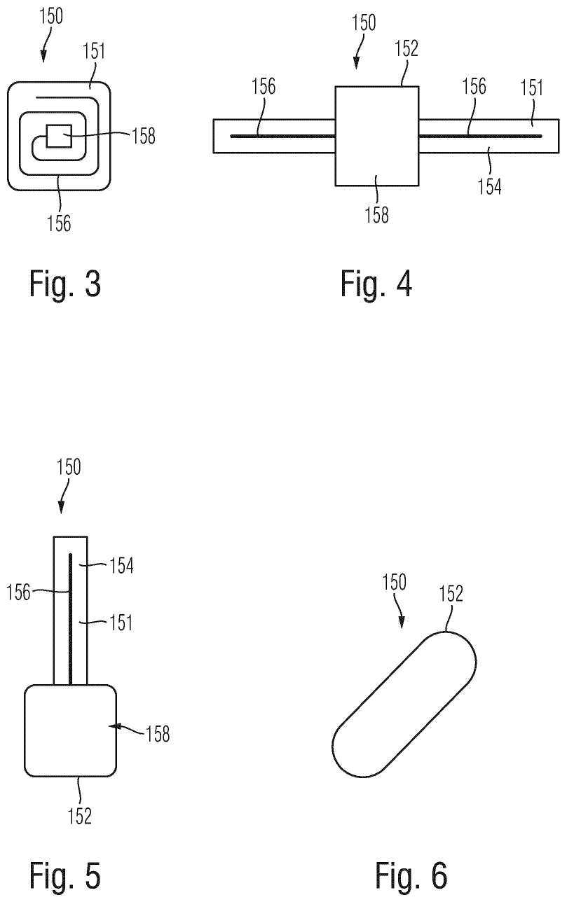

In FIGS. 3-6 there are illustrated numerous examples of an electronic identifier 150. The electronic identifier 150 comprises a substrate 151. The substrate 151 may be a rather rigid substrate 151. The substrate 151 provides a mount and a mechanical support for the electronic identifier 150. Typically, the electronic identifier 150 comprises a processing unit 158. The processing unit 158 may comprise a microprocessor or a microcontroller. The electronic identifier 150 further comprises an antenna 156 electrically connected to the processor 158.

The electronic identifier 150 may be implemented as a passive or as an active RFID tag. With some examples the substrate 151 is a transparent substrate 154. In the example of FIGS. 4 and 5, the substrate 151 laterally protrudes from the processing unit 158. Here, the substrate 151 may be rigid and transparent. The substrate 151 protruding laterally from the processing unit 158 provides support for the antenna 156.

With these or other examples the transparent substrate 154 is flexible. Here, the substrate 154 may comprise a flexible foil. With some examples, the substrate 154 transparent. With some examples the substrate 151 is opaque. With some examples the substrate is transparent and flexible. With some examples the substrate is rigid and opaque. With other examples the substrate is flexible and opaque.

The processing unit 158 as illustrated in FIGS. 3-5 may be provided by a microchip or may comprise a microchip. The processing unit 158 may comprise a rigid housing 152. Moreover, the processing unit 158 may be embedded or encapsulated in the rigid housing 152. In this way, a dimensionally stable electronic identifier 150 can be provided. With the example of FIG. 3, the antenna 156 is coiled around the processing unit 158. Here, the processing unit 158 and the coiled antenna 156 are provided and/or arranged on a common substrate 151.

An elongated design or configuration of the electronic identifier as illustrated in FIGS. 4 and 5 may be particularly adapted or suitable for assembly in an elongated receptacle 120 as illustrated in FIG. 7. There, the mount 110 for the electronic identifier 150 is located on an outside of the barrel 101. The mount 110 and hence the receptacle 120 extends in longitudinal direction from the distal end 106 towards and/or until the proximal end 108 of the barrel 101. An outside surface 126 of the receptacle 120 facing radially outwardly may be provided with a transparent, semitransparent or frosted window 127. The window 127 may be provided with a scale 128 extending in the longitudinal direction. In this way and with a transparent sidewall of the barrel 101 the content of the barrel 101 can be visually inspected.

The substrate 151, 154 is either flexible or rigid. The substrate 154 is typically transparent. On the substrate 154 or encapsulated in the substrate 154 there is provided the electronic identifier 150. Hence, the processing unit 158 and the antenna 156 are provided on or in the transparent substrate 154. The substrate may be also provided with a scale 128 visible through the window 127 of the receptacle 120. Here, the scale 128 may be exclusively provided on the electronic identifier 150. Insofar the electronic identifier 150 provides a twofold function. On the one hand it serves to encode or to label the medicament container 100 in an electronically readable way. On the other hand it may provide a visual scale to indicate to the filling level of the medicament container 100.

The substrate 154 with the processing unit 158 and the antenna 156 thereon is generally insertable through the insert opening 122. The insert opening 122 faces in proximal direction 3. Accordingly, the electronic identifier 150 is insertable into the receptacle 120 through the insert opening 122 in distal direction 2 until it reaches a final assembly configuration. The sidewall of the receptacle 120 may be provided with at least one retention feature 130 such as at least one catch element 132 as schematically illustrated and described in connection with FIG. 9.

Alternatively or additionally, the insert opening 122 of the receptacle 120 may be closed or sealed. The insert opening one 4 and 22 may be closed or covered by the body 10 of the injection device 1. In a final assembly configuration a distal abutment face 19 provided at the distal end of the body 10 may abut with and may effectively cover or close the insert opening 122. Such an abutment or closing configuration may be reached when the mechanical interface 112 of the medicament container 100 engages the mechanical counter interface 32 of the body 10.

With other examples of the electronic identifier 150 the electronic identifier 150 is encapsulated or wrapped inside a rigid housing 152. The housing 152 may resemble a pill and may thus comprise a pill-like design. Here, the electronic identifier 150 is somewhat miniaturized. This allows for an easy and space-saving arrangement or attachment of the electronic identifier 150 to the medicament container. Moreover, the rigid housing 152 may be structurally stable. This allows to attach and/or to fix the electronic identifier 150 in a press-fit configuration inside the receptacle 120 or to arrange the electronic identifier 150 in a press-fit to the mount 110.

With a further example as illustrated in FIGS. 10-12 the mount 110 is arranged proximally and/or longitudinally adjacent to the mechanical interface 112 of the medicament container 100. Here, the mount 110 and the receptacle 120 are located adjacent a stepped down portion of the barrel 101 towards the proximal end 108. The stepped down portion forms an insert section 115 at the proximal end 108 of the medicament container 100. The insert section 115 is configured to be inserted into the receptacle 35 provided at the distal end of the body 10.

As indicated in FIG. 11 the mount 110 and the receptacle 120 protrude radially outwardly from the outside surface of the barrel 101. The insert opening 122 faces in proximal direction 3. An abutment face 124 may laterally enclose the insert opening 122. The electronic identifier 150 is insertable into the insert opening 122 and hence into the receptacle 120 along the distal direction 2. Once the electronic identifier 150 has been appropriately assembled inside the receptacle 120 the medicament container 100 will be assembled in a releasable or non-releasable way with the distal end of the body 10.

For this, the insert section 115 and hence the mechanical interface 112 of the medicament container 100 or of the barrel 101 is inserted in longitudinal proximal direction 3 into the receptacle 35 of the body 10. The receptacle 35 comprises at least one or several radially inwardly protruding counter fastening features 14 configured to engage with correspondingly or complementary shaped fastening features 114 on the outside surface of the sidewall of the mechanical interface 112. As indicated in FIG. 10, the at least one fastening feature 114 comprises a radial recess on the outside surface of the sidewall of the mechanical interface 112.

As illustrated in FIG. 12 and when a final assembly configuration has been reached the distally facing abutment face 19 of the body 10 abuts with the proximally facing abutment surface 124 of the mount 110 and hence of the receptacle 120. In this way and upon establishing a mutual interconnection of the medicament container 100 with the body 10 the receptacle 120 accommodating or holding the electronic identifier 150 is effectively closed.

The presently illustrated mutual mechanical interconnection between the medicament container 100 and the body 10 is only exemplary. With other examples the insert section 115 is provided with a radially outwardly extending protrusion configured to engage with a radial recess on an inside surface of the receptacle 35.

With other examples it is the medicament container 100 that comprises a receptacle configured and sized to receive a complementary shaped insert section at the distal end of the body 10.

With further or other examples the snap fit connection as provided by the fastening feature 114 and the counter fastening feature 14 may be replaced by a friction fit, by an adhesive attachment and/or by a welding of the medicament container 100 to the body 10.

The mechanical interface 112 may be optionally provided with an anti-rotating feature 117 that mates with a correspondingly or complementary-shaped counter anti-rotating feature of the mechanical counter interface 32. The anti-rotating feature 117 may comprise one of a longitudinal slit, groove, rib or protrusion configured to engage with a correspondingly or complementary shaped rib, protrusion, slit or groove of the body 10. In this way the mechanical engagement of the medicament container 100 and the body 10 can be only provided when the medicament container 100 is in at least one of numerous predefined angular orientations with regards to the body 10.

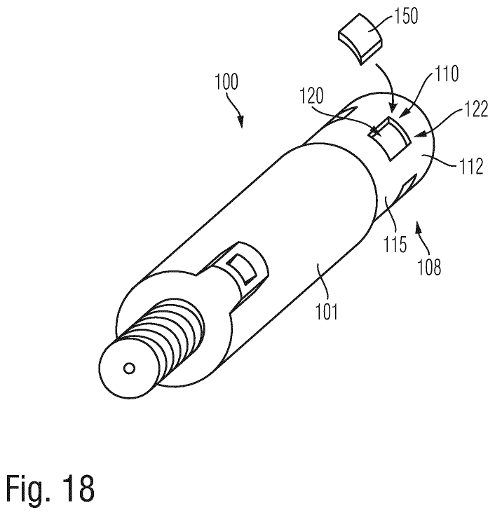

In the example of FIG. 18 the receptacle 120 and hence the mount 110 for the electronic identifier 150 is located in the mechanical interface 112. Also here, the mechanical interface 112 provided at the proximal end of the medicament container 100 comprises an insert section 115 configured and sized for insertion into the receptacle 35 provided at a distal end of the body 10.

Here and contrary to the example of FIGS. 10-12 the receptacle 122 is open towards a radial outer direction. Hence, the electronic identifier 150 is to be inserted radially inwardly into the receptacle 120. When reaching a final assembly configuration, in which the insert section 115 is received inside the receptacle 35 of the body 10 the insert opening 122 of the receptacle 120 is effectively closed and the electronic identifier 150 is mechanically secured in the interface between the body 10 and the medicament container 100.

As schematically illustrated in FIG. 13 the electronic identifier 150 may not only comprise a processing unit 158 and an antenna 156. The electronic identifier 150 may comprise an electronic storage 159 and may optionally also comprise an electric energy reservoir 157. The processing unit 158 and the electronic storage 159 may be integrated in one and the same microchip. The electronic storage 159 may be implemented as a read only storage. With some examples the electronic storage 159 is implemented as a read-write storage.

The electronic storage may be configured to store data being indicative of at least one of a manufacturing day, a manufacturing time, a best before date, a manufacturing lot, an individual manufacturing ID, a manufacturer information such as a manufacturer ID, a manufacturing location. The data stored in the electronic storage 159 may comprise or represent medicament container-relevant information during manufacturing, during assembly, during shipment, during storage, during retail and/or during use of the medicament container. The processing unit 158 of the electronic identifier 150 may be configured to retrieve and to process data from an external electronic device and to write the respective data in the electronic storage 159. Upon request and/or at the end of life cycle of the medicament container and100 and/or of the electronic identifier 150 respective information or data stored in the electronic storage 159 may be retrieved and collected by the external electronic device.

Some examples the electronic identifier 150 are void of an electric energy reservoir 157. The electronic identifier 150 may be then implemented as a passive RFID tag. Electric energy for operating of the processing unit 158 may be withdrawn from an interrogation RF field received by the antenna 156. With other examples the electronic identifier 150 comprises an electric energy reservoir 157. The electric energy reservoir 157 may comprise a battery, e.g. a rechargeable battery.

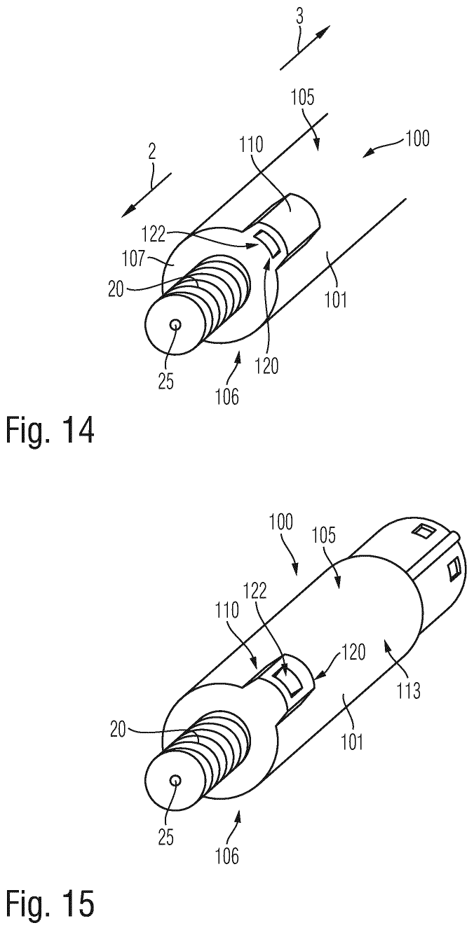

With the example of FIG. 14, the mount 110 is provided at a distal end 106 or the barrel 101. Here, the receptacle 120 comprises an insert opening 122 facing in distal direction 2. Accordingly, the electronic identifier 150 is insertable into the receptacle 120 along the proximal direction 3. The receptacle 120 and/or the insert opening 122 may flush with a stepped down shoulder portion 107 or neck portion of the medicament container 100, from which the threaded portion 20 may protrude in distal direction 2. The insert opening 122 and/or a sidewall of the receptacle 120 may flush with the shoulder portion 107 or neck portion of the medicament container 100. This allows for an easy, intuitive and straightforward insertion of the electronic identifier 150 into the receptacle 120.

In the further example of FIG. 15, the mount 110 and hence the receptacle 120 are also located at or near the distal end 106 of the medicament container 100. But there the insert opening 122 faces in a radial outward direction. Accordingly, the electronic identifier 150 is insertable into the receptacle 150 in a radial inward direction.

With both examples as illustrated in FIGS. 14 and 15 the position of the mount 110 and of the receptacle 120 at or near the distal end 106 is of particular benefit when the sidewall of the barrel 101 is made of a transparent material or when the sidewall comprises at least a transparent portion 113. In this way and irrespective on whether the receptacle 120 and the electronic identifier 150 are of transparent or opaque an obstructed view of the medicament located in the interior volume 105 of the barrel 101 is provided.

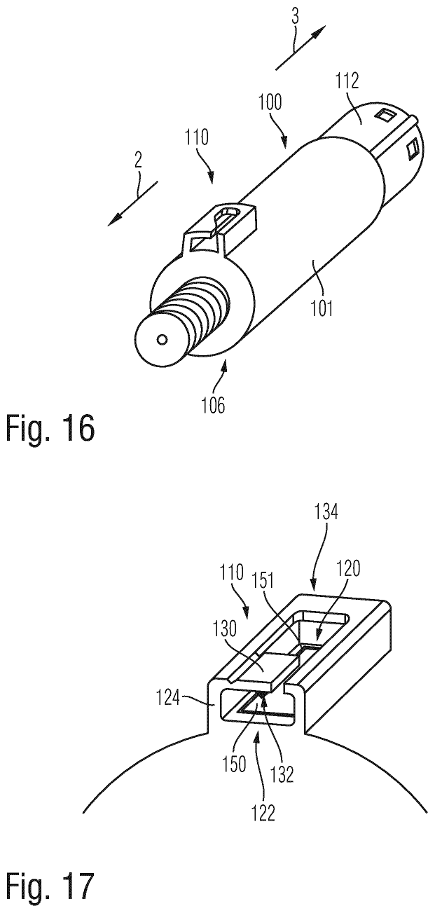

In the further example of FIGS. 16 and 17 the mount 110 is also provided at or near a distal end 106 of the medicament container 100. The mount 110 slightly protrudes radially from an outside surface of the tubular shaped barrel 101. The mount 110 comprises a receptacle 120 with an insert opening 122 facing in distal direction 2. There is further provided a retention feature 130 at an upper or radial outer edge of the insert opening 122. The retention feature 130 comprises a radially inwardly protruding catch element 132 configured to engage with the electronic identifier 150.

Here, the electronic identifier 150 comprises a rigid substrate 151. The electronic identifier 150 may resemble or may form a rigid card of chip and may be configured for insertion into the insert opening 122 along the proximal direction 3. The retention feature 130 may be flexible in radial direction. The retention feature 130 may be configured to exert a radially inwardly directed clamping force onto the substrate 151 and/or on an encapsulation or rigid housing 152 of the electronic identifier 150. In other words, the retention feature 130 may comprise a hold-down clamp configured to clamp and to fix the electronic identifier 150 inside the receptacle 120. The retention feature 130 may be pre-tensed radially inwardly. It may be resiliently deformable radially outwardly against an intrinsic restoring force.

The upper or radially outwardly facing sidewall of the receptacle 120 comprises a recess or an aperture 134. The recess or aperture 134 in the receptacle 120 provides and enables visual inspection of the electronic identifier 150. Here, a user may easily control, the presence and/or correct assembly of the electronic identifier 150 when attached to the mount 110 and when arranged inside the receptacle 120.

The medicament container 100 as illustrated in FIGS. 1-18 may be implemented as a primary medicament container. Here, the barrel 101 of the medicament container 100 is configured and intended to accommodate the liquid medicament directly. Hence, an inside facing surface of the sidewall of the barrel 101 gets in direct contact with the liquid medicament as the interior volume 105 is filled with the liquid medicament.

With further examples of the present disclosure the medicament container 100 as illustrated in FIGS. 1-18 is implemented as a secondary medicament container. A secondary medicament container does not get in direct contact with the liquid medicament. Rather the secondary medicament container is configured to receive and/or to accommodate a primary medicament container, wherein the primary medicament container is filled or provided with the liquid medicament.

Insofar, all features as described above with regards to the arrangement and fixing of an electronic identifier 150 to or into a mount 110 of a primary medicament container 100 equally apply to a secondary medicament container.

A primary medicament container 100 may be implemented as a cartridge, as a carpule, as a vial or as a syringe. A secondary medicament container may be implemented or configured as a cartridge holder, as a carpule holder or as a syringe holder. When the medicament container is implemented as a secondary medicament container configured to receive or to accommodate a primary medicament container it may be sufficient when only one of the primary medicament container and the secondary medicament container is provided with a mount 110 for the electronic identifier 150.

With some examples, both, the primary medicament container and the secondary medicament container may be each provided with a mount for an electronic identifier. At least one or both of these mounts may be then equipped with such an electronic identifier 150.

An example of such a secondary medicament container 300 is provided in FIG. 30. The secondary medicament container 300 comprises a tubular-shaped barrel 301. The barrel 301 is of elongated shape. Its cylindrical axis extends along the longitudinal direction. The barrel 301 comprises a distal end 306 at one longitudinal end. The distal end 306 is provided with an outlet 302. An oppositely located longitudinal end is a proximal end 308. The barrel 301 defines an interior volume 305. The interior volume 305 is accessible through the outlet 302.

The outlet 302 comprises an aperture through which the outlet 102 of the primary medicament container 100 as illustrated in any of the FIGS. 1-18 may extend. The aperture at the outlet 302 is sized to provide access to the primary medicament container 100 located inside the secondary medicament container 300. A needle hub 22, an injection needle 24 and/or some other type of liquid medicament guiding structure, such as an infusion line may at least partially reach through the aperture of the outlet 302 in order to establish a fluid coupling between the primary medicament container 100 and at least one of an infusion line and an injection needle 24.

The secondary medicament container 300 is provided with a mount 310 for the electronic identifier 150. In the example as illustrated in FIG. 30, the mount is provided at or near a proximal end 308 of the barrel 301. It may comprise a receptacle 320 and an insert opening 322 that are open towards the proximal direction 3. The secondary medicament container 300 as illustrated in FIG. 30 may also comprise a mechanical interface at its proximal end 308 in order to attach or to connect the secondary medicament container 300 to a drug delivery device, e.g. to an injection device 1.

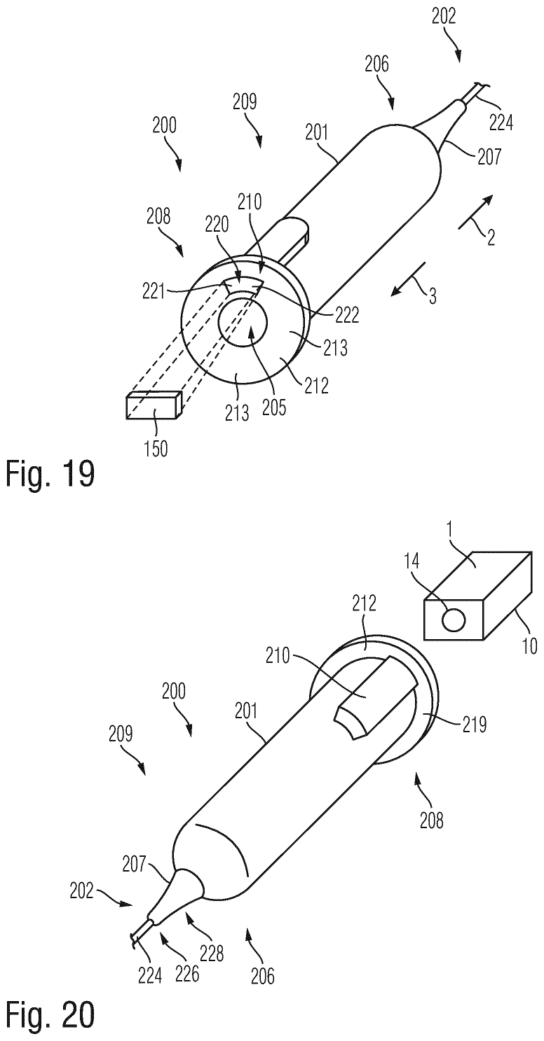

In FIGS. 19 and 20 another example of a medicament container 200 is illustrated. This medicament container 200 comprises a tubular-shaped barrel 201. The medicament container 200 is implemented as a primary medicament container. The medicament container 200 comprises an interior volume 205 configured to directly receive and/or to hold or to accommodate the liquid medicament. The barrel 201 comprises a distal end 206 and an oppositely located proximal end 208. Towards or near the distal end 206 the tubular shaped barrel 201 features a radially stepped down shoulder portion 207. The shoulder portion 207 may form or constitute the distal end 206 of the barrel 201. The distal end 206 of the barrel 201 is provided with an outlet 202 for the liquid medicament. With some examples the outlet 202 is provided with an injection needle 224. As illustrated in FIGS. 19 and 20 the injection needle 224 is directly connected, e.g. welded or injection molded in the outlet 202. The injection needle 224 extends through the outlet 202 or may contribute to the outlet 202. A proximal end of the injection needle 224 is located inside the interior volume 205. A distal end of the injection needle 224 protrudes from the barrel 201 in distal direction.

With some examples the outlet 202 of the barrel 201 is provided with a container interface 228 correspondingly or complementary shaped to a needle interface 226 of the injection needle 224. The needle interface 226 and the container interface 228 may comprise mutually corresponding standardized adapters or connectors by way of which a liquid tight releasable connection can be established between the injection needle 224 and the outlet 202 of the barrel 201.

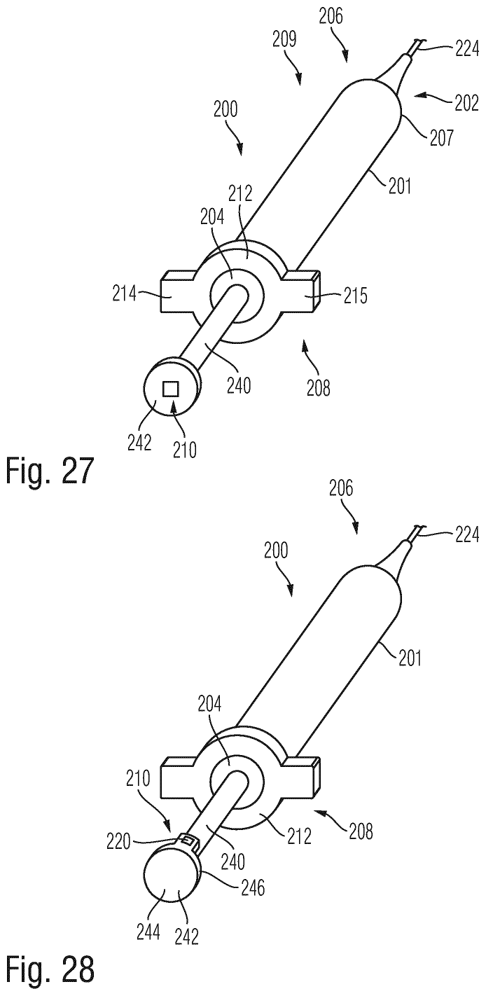

The medicament container 200 as illustrated in FIGS. 19 and 20 is implemented as a syringe 209. The syringe 209 may be implemented as a disposable syringe. It may be intended for a single or one-time use. In FIGS. 19 and 20 the syringe 209 is illustrated without a piston 204. Such a piston 204 is illustrated in connection with FIGS. 27-29. Typically and when appropriately filled with the liquid medicament the interior volume 205 of the barrel 201 is sealed towards the proximal direction 3 by such a piston 204.

The piston 204, typically made of an elastomeric material or featuring at least an elastomeric coating is slidably displaceable inside the hollow cylindrical structure of the barrel 201. By displacing the piston 204 in distal direction 2 a respective amount of the liquid medicament can be expelled through the outlet 202 and hence through the injection needle 224.

The barrel 201 may comprise an injection moldable plastic material. As further illustrated in FIGS. 19 and 20 the barrel 201 is provided with a mount 210 on the outside surface of the barrel 201. The mount 210 comprises a receptacle 222 with an insert opening 222 that is accessible from the proximal end 208 of the barrel 201. The insert opening 222 is open towards the proximal direction 3. Alternatively, the insert opening 222 is open towards the distal direction 2 or towards a tangential direction.

The receptacle 220 and hence the mount 210 for the electronic identifier 150 is located at or near the proximal end 208 of the medicament container 200. At the proximal end 208 the barrel 201 comprises a radially widened flange 212. In the example as illustrated in FIGS. 19 and 20 the flange 212 is of circular or elliptic shape. The flange 212 protrudes radially outwardly from the cylindrical sidewall of the barrel 201. The flange 212 comprises a proximal face 213. The receptacle 220, e.g. implemented as a pocket recess 221, is flush with the proximal face 213 of the flange 212. The flange 212 serves as a finger flange thus allowing to grip and to hold the syringe 209 by e.g. an index finger and a middle finger.

The electronic identifier 150 can be mounted inside the receptacle 220 in a way as described above in connection with the receptacle 120 of FIG. 6-18. Also here, the electronic identifier 150 may be implemented in accordance to any of the examples as described above in connection with FIGS. 3-6.

The receptacle 220 and hence the pocket recess 221 is accessible at the proximal end 208 of the medicament container 200. The syringe 209 and hence the medicament container 200 may be implemented for manual use, e.g. by a physician or by medical staff. With some examples the medicament container 200 may be configured for use with a drug delivery device 1 as indicated in FIG. 20.

The medicament container 200, hence the syringe 209, may be configured to operate with a drug delivery device 1 implemented as a syringe driver or syringe pump. For this, the drug delivery device 1 comprises a housing 10 provided with a counter fastening feature 14 configured to engage with a fastening feature 219 of the medicament container 200. The fastening feature 219 may be provided by the radially outwardly protruding flange 212. Moreover, also a combination of the stepped down shoulder portion 207 and the proximal end 208 of the barrel 201 may equally serve as or constitute to the fastening feature 219 that allows to fit or to fix the medicament container 200 to a suitable fastening structure 14 or receptacle of a drug delivery device 1.

The drug delivery device 1 may be provided with a transducer and an antenna to establish a wireless communication link between the drug delivery device 1 and the electronic identifier 150. With some examples the drug delivery device 1 may be provided with a logic unit and/or with a processor by way of which electronic data stored in the electronic identifier 150 can be read out and processed in order to control operation of the drug delivery device 1.

With some examples the receptacle 220 as provided in the flange 212 of the syringe 209 may be closed or covered by a counter fastening feature 14, by the housing 10 or by some other part of the drug delivery device 1 when the medicament container 200 is appropriately connected or fastened to the drug delivery device 1. In this way, the electronic identifier 150 can be securely and non-detachably fixed inside the mount 210 at least as long as the medicament container 200 is mechanically engaged with the drug delivery device 1.

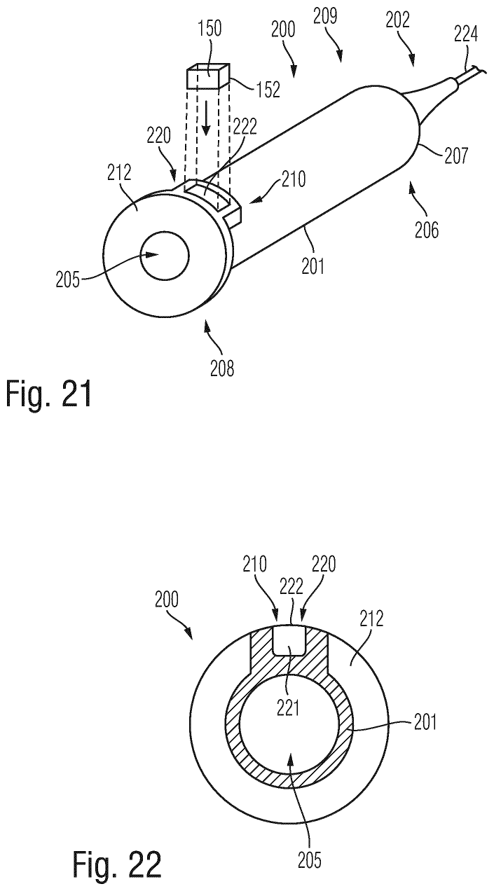

With the example of FIGS. 21 and 22 the mount 210 for the electronic identifier 150 is also provided at the proximal end 208 of the barrel 201. Here and in contrast to the example of FIGS. 19 and 20 the receptacle 220 is provided on the outer circumference of the flange 212. The insert opening 222 is open towards a radial outer direction. Consequently, the electronic identifier 150 is insertable into the receptacle 220 or pocket recess 221 along a radial direction, hence radially inwardly.

As can be seen from the cross-section of FIG. 22, the receptacle 220 is implemented as a pocket recess 221. A bottom portion of the pocket recess 221 is provided or formed by the tubular-shaped sidewall of the barrel 201. The sidewalls of the receptacle 220 protrude radially outwardly from an outside wall of the barrel 201. At least one axial sidewall of the receptacle 220 is provided by the radially outwardly extending flange 212. Hence, the receptacle is delimited in proximal direction by the flange 212.

Depending on the specific use and/or depending on a specific mechanical connection, e.g. to a drug delivery device 1, the insert opening 222 may be also covered and closed by the housing 10, a counter fastening feature 14 or some other part of the drug delivery device 1.

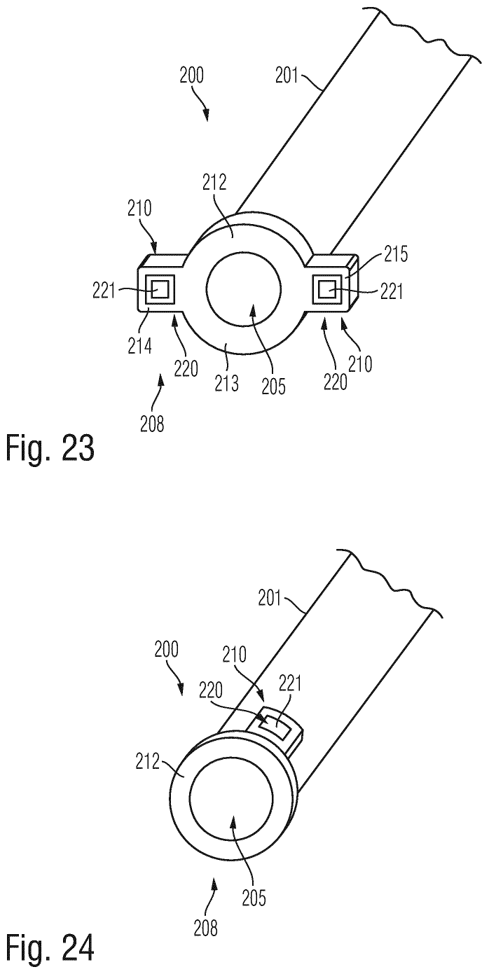

In the further example as illustrated in FIG. 23, the flange 212 is provided with a first radially outwardly protruding flange section 214 and with a second radially outwardly protruding flange section 215. The first and the second flange sections 214, 215 protrudes radially outwardly from the rather circularly shaped flange 212 at the proximal end 208 of the barrel 201. The first flange section 214 and the second flange section 215 are located on the diametrically opposite sides of the flange 212. Since the first and second flanges sections 214, 215 feature a comparatively large extension in radial direction they are particularly suitable to provide a receptacle 220 and/or a pocket recess 221 to receive or to accommodate the electronic identifier 150.

Moreover, there may be even provided two separate electronic identifiers in each one of the receptacles 220 of the first flange section 214 and the second flange section 215, respectively. As illustrated in FIG. 23 the receptacles 220 and hence the pocket recesses 221 are provided in the proximal face 213 of the flange 212, in particular in the proximal face 213 of the first and second flange sections 214, 215. Also here and when fixed to an injection device 1, the pocket recesses 221 may be suitably covered and closed by e.g. a housing component or by a fastening feature of the drug delivery device 1.

In FIG. 24 another example of a syringe type medicament container 200 is illustrated. Here, the receptacle 220 is open towards the radial direction and adjoins the flange 212. The mount 210 for the electronic identifier 150 protrudes radially outwardly from the barrel 201 of the medicament container 200. It is located adjacent to the radially outwardly extending flange 212.

With the examples of FIGS. 23 and 24, the electronic identifier 150 may be insert molded in the respective mount 210 or receptacle 220.

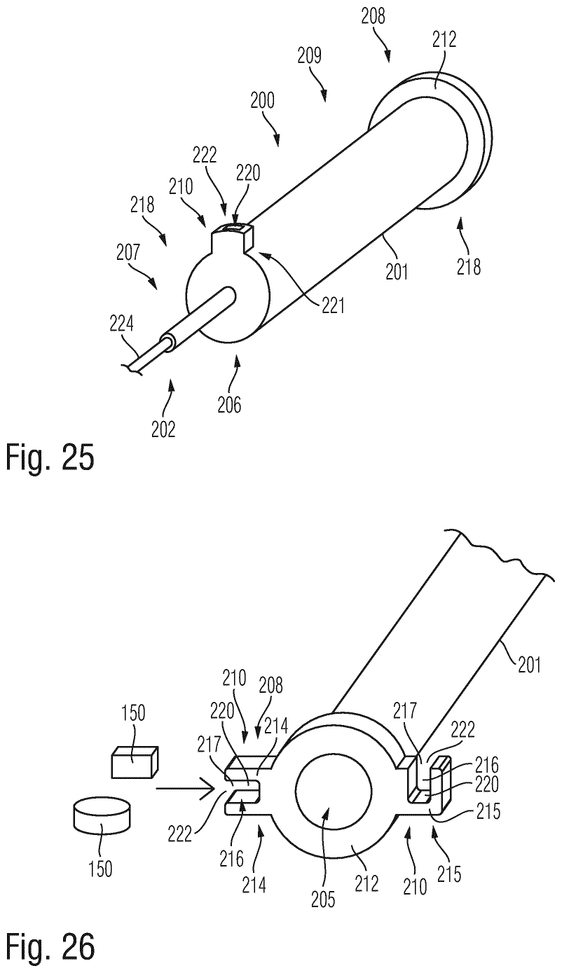

With the further example of FIG. 25 the mount 210 is provided at the distal end 206 of the barrel 201. There, the mount 210 protrudes radially from the tubular portion of the barrel 201. The mount 210 comprises a receptacle formed as a pocket recess 221 having an insert opening 222 that is accessible from the radial direction. A distal end of the mount 210 may flush with a distal end of the tubular shaped portion of the barrel 201. A nozzle or spout may protrude distally from the distal end of the barrel 201 and may provide or contribute to the outlet 202. As illustrated in FIG. 25 the outlet 202 of the barrel 201 is provided with an injection needle 224.

Here, the distal end face of the tubular shaped portion of the barrel 201 as well as a proximal end face of the barrel 201 and/or of the flange 212 may form or constitute a mechanical connection interface 218 by way of which the syringe 209 can be fixed or attached to a drug delivery device 1, such as a syringe driver or syringe pump.

The presently illustrated configuration of the mount 210 is by no way limiting. It is also conceivable, that the mount 210 is provided at or near the distal end 206 of the barrel 201 in such a way, that the receptacle 220 and/or its insert opening 222 is facing in circumferential or tangential direction, in proximal direction 3 or in distal direction 2.

In the further example as illustrated in FIG. 26 the mount 210 for the electronic identifier 150 is provided at or near the proximal end 208 of the barrel 201. Here, the flange 212 comprises a first flange section 214 and a second flange section 215. The first flange section 214 comprises a receptacle 220 formed as a slit 216 or as an aperture 217 in the radial outside edge of the first flange section 214. The slit 216 or aperture 217 is shaped to receive the electronic identifier 150 or a rigid housing 152 of an electronic identifier 150. The slit 216 or aperture 217 may be sized to form a clamp connection for the electronic identifier 150.

The second flange section 215 is also provided with a slit 216 or aperture 217. The slit 216 or aperture 217 is open towards the tangential edge and/or towards the tangential direction of the flange 212. It may be likewise suitable to perform a clamp connection with the electronic identifier 150 or with a rigid housing 152 thereof. It is generally conceivable, that the medicament container 200 comprises only one of a slit 216 or aperture 270 as presently illustrated with the example of the first and the second flange sections 214, 215 of the barrel 201 of FIG. 26.

In FIG. 27 another example of a medicament container 200 implemented as a syringe 209 is illustrated. This syringe 209 is provided with a piston 204 forming a proximal seal of the interior volume 205 of the tubular shaped barrel 201. The piston 204 is in sealing engagement, hence in a fluid tight engagement with the sidewall of the barrel 201. A proximal face of the piston 204 is connected or is connectable to a plunger rod 240. A proximal end of the plunger rod 240 is provided with a radially widened flange portion 242. Here, it is the flange portion 242 that is provided with the mount 210 for the electronic identifier 150. The electronic identifier 150 may be insert molded in the flange portion 242.

Typically, at least the flange portion 242 and the plunger rod 240 are provided as a single pieced injection molded plastic component. Optionally, also the piston 204 may be integrally formed with the plunger rod 240. For providing a sufficient sealing capability the piston 204 may be at least coated with an elastomeric material. The radially widened and comparatively large planar shape of the flange portion 242 is particularly suitable to form the mount for the electronic identifier or to receive the electronic identifier therein.

With the further example of FIG. 28, the mount 210 for the electronic identifier is provided in a sidewall 246 of the flange portion 242. Here, the flange portion 242 comprises a planar shaped proximal end face 244. The mount 210 is located distally adjacent to the proximal end face 244. The mount 210 and hence its receptacle 220 is accessible in radial direction. Hence, the electronic identifier 150 may be inserted into the receptacle 220 radially inwardly.

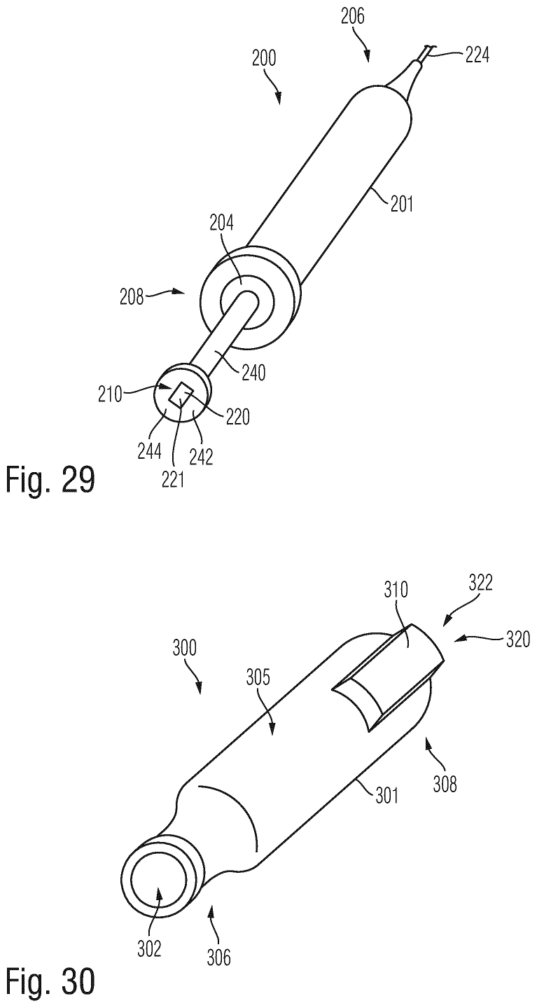

In the further example of FIG. 29, the mount 210 is provided in the proximal end face 244 of the flange portion 242 of the plunger rod 240. It may flush with the end face 244. When the electronic identifier 150 is assembled inside the receptacle 220 it may not protrude from the receptacle 221. The receptacle 220 typically comprises a pocket recess 221 sized to receive the entirety of the electronic identifier 150 and/or of its rigid housing 152.

Generally and with nearly all mounts 210 and receptacles 220 as illustrated and described above in connection with FIGS. 19-30 the mount 210 may be provided with such retention features 130 or with such catch elements 132 as described above in connection with FIGS. 8 and/or 9. Respective features and benefits described in connection with the mounting and securing of the electronic identifier 150 in or to the mount 110 equally apply to the respective mounts 210 as described above in connection with FIGS. 19-30.

### LIST OF REFERENCE NUMBERS

1 injection device

2 distal direction

3 proximal direction

4 dose incrementing direction

5 dose decrementing direction

6 housing

10 body

11 trigger

12 dose dial

13 dosage window

14 counter fastening feature

15 needle assembly

16 inner needle cap

17 outer needle cap

18 protective cap

19 abutment face

20 threaded portion

22 needle hub

24 injection needle

25 aperture

26 piston rod

28 pressure piece

30 drive mechanism

32 counter interface

35 receptacle

100 medicament container

101 barrel

102 outlet

104 piston

105 interior volume

106 distal end

107 shoulder portion

108 proximal end

110 mount

112 interface

113 transparent portion

114 fastening feature

115 insert section

117 anti-rotating feature

120 receptacle

122 insert opening

124 abutment face

126 outside surface

127 window

128 scale

130 retention feature

132 catch element

134 aperture

150 electronic identifier

151 rigid substrate

152 rigid housing

154 transparent substrate

156 antenna

157 electric energy reservoir

158 processing unit

159 electronic storage

200 medicament container

201 barrel

202 outlet

204 piston

205 interior volume

206 distal end

207 shoulder portion

208 proximal end

209 syringe

210 mount

212 flange

214 flange section

213 proximal surface

215 flange section

216 slit

217 aperture

218 interface

219 fastening feature

220 receptacle

221 pocket recess

222 insert opening

224 injection needle

226 interface

228 interface

240 plunger rod

242 flange portion

244 end face

246 sidewall

300 container

301 barrel

302 outlet

305 interior volume

306 distal end

308 proximal end

310 mount

320 receptacle

322 insert opening

1. A medicament container (100; 200) for a liquid medicament, the medicament container comprising:
a barrel (101; 201) defining a longitudinal direction (2, 3) and comprising an interior volume (105; 205) to accommodate the liquid medicament, the barrel (101; 201) comprising a distal end (106; 206) and a proximal end (108; 208), the barrel (101; 201) comprising an outlet (102: 202) for the liquid medicament at the distal end (106; 206),
a mount (110; 210) for an electronic identifier (150),
an electronic identifier (150) engaged with the mount (110; 210) and attached to the barrel (101; 201) via the mount (110; 210).

2. The medicament container (100; 200) according to claim 1, wherein the mount (110; 210) comprises a receptacle (120; 220) with an insert opening (122; 222) sized to receive the electronic identifier (150).

3. The medicament container (100; 200) according to any one of the preceding claims, wherein the mount (110;210) comprises at least one retention feature (130) configured to fix the electronic identifier (150) in the receptacle (120; 220), wherein the retention feature (130) being one resiliently deformable and pivotally supported on or in the receptacle (120; 220).

4. The medicament container (100; 200) according to claim 3, wherein the at least one retention feature (130) comprises at least one catch element (132) configured to engage with the electronic identifier (150) when the electronic identifier (150) is arranged in a predefined assembly position inside the receptacle (150).

5. The medicament container (100; 200) according to any one of the preceding claims, wherein the electronic identifier (150) comprises at least one of a rigid substrate (151) and a rigid housing (152) and wherein at least one of the rigid substrate (151) and the rigid housing (152) is mechanically engageable with the mount (110).

6. The medicament container (100; 200) according to claim 5, wherein at least one of the rigid substrate (151) and the rigid housing (152) is mechanically clamped and/or mechanically squeezed in the mount (110; 210) or at the mount (110; 210).

7. The medicament container (100; 200) according to any one of the preceding claims, wherein the electronic identifier (150) comprises a transparent substrate (154).

8. The medicament container (100; 200) according to any one of the preceding claims, wherein the electronic identifier (150) is a wireless electronic identifier, wherein the electronic identifier (150) comprises an antenna (156) and a processing unit (158) electrically connected to the antenna (156).

9. The medicament container (100; 200) according to any one of the preceding claims, wherein the barrel (101) comprises a mechanical connection interface (112; 218) configured to connect the barrel (101) to a body (10) of an injection device (1), wherein the mechanical connection interface (112; 218) comprises at least one fastening feature (114; 219) configured to mechanically engage with a complementary-shaped counter fastening feature (14) of the body (10) of the injection device (1).

10. The medicament container (100; 200) according to claim 9, wherein the mount (110; 210) is arranged on an outside surface of the barrel (101, 201) and wherein the electronic identifier (150) is fixed to the mount (110) by the body (10) of the injection device (1) when the at least one fastening feature (114; 219) is engaged with the counter fastening feature (14).

11. The medicament container (100; 200) according to claim 2 and any one of the preceding claim 9 or 10, wherein the insert opening (122; 222) of the receptacle (120; 220) is covered or closed by a component of the injection device (1) when the at least one fastening feature (114;

219. is engaged with the counter fastening feature (14).

12. The medicament container (100; 200) according to claim 11, wherein the barrel (101; 201) comprises an elongated structure defining a longitudinal direction (2, 3) and a radial direction, wherein the insert opening (122; 222) of the receptacle (120; 220) is open towards the longitudinal direction (2, 3) or wherein the insert opening (122; 222) of the receptacle (120; 220) is open towards the radial direction.

13. The medicament container (100; 200) according to any one of the preceding claims, wherein the barrel (101; 201) is made of a plastic material.

14. The medicament container (200) according to any one of the preceding claims, wherein the medicament container comprises a syringe (200) and wherein the barrel (201) is a syringe barrel.

15. The medicament container according to any one of the preceding claims, wherein the medicament container (100; 200) is filled with the liquid medicament.

16. A drug delivery device for administering a dose of a liquid medicament, the injection device comprising a housing (6) and a drive mechanism (30),
the housing comprising a body (10) and a medicament container (100; 200) according to any one of the preceding claims,
wherein the drive mechanism (30) arranged or fixed inside the body (10) comprises a driver (26) operable to engage with the medicament container (100; 200) for expelling or withdrawing the liquid medicament from the medicament container (100).
